(19) United States
(12) Patent Application Publication
(10) Pub. No.: US 2020/0138410 A1
(43) Pub. Date: May 7, 2020

(54) ULTRASONIC DIAGNOSTIC APPARATUS

(71) Applicant: Konica Minolta, Inc., Tokyo (JP)

(72) Inventors: Akihiro Kawabata, Tokyo (JP); Tomohito Sakai, Yokohama-shi (JP)

(21) Appl. No.: 16/597,195

(22) Filed: Oct. 9, 2019

(30) Foreign Application Priority Data
Nov. 2, 2018 (JP) 2018-207150

(51) Int. Cl. A61B 8/00; A61B 8/08

(52) U.S. Cl. CPC A61B 8/467; A61B 8/488; A61B 8/485; A61B 8/461; A61B 8/486; A61B 8/5246; A61B 8/5207

(57) ABSTRACT

An ultrasonic diagnostic apparatus includes: a first hardware processor that generates ultrasonic image data, based on a reception signal obtained by an ultrasonic probe that transmits and receives an ultrasonic wave; a storage that stores first image parameters associated with a depth corresponding to an ROI; and a second hardware processor that causes the first hardware processor to generate the ultrasonic image data, based on the first image parameters.

### BACKGROUND

### Technological Field

The present invention relates to an ultrasonic diagnostic apparatus.

### Description of the Related Art

According to ultrasonic diagnosis, situations of a heart, a fetus or the like are obtained as ultrasonic images through a simple operation of bringing an ultrasonic probe into contact with the body surface or an inner surface of a body cavity. Since the safety is high, tests can be repetitively performed. Ultrasonic diagnostic apparatuses used to perform such ultrasonic diagnosis have been known. Ultrasonic waves are transmitted from an ultrasonic probe to a subject, reflected ultrasonic waves are received by the ultrasonic probe and converted into an electric signal (hereinafter called a reception signal), and various processes are applied to the reception signal, thereby obtaining ultrasonic image data.

In generation of the ultrasonic image data, various image parameters are set, which can variously affect the ultrasonic image data to be generated. For example, as for generation of ultrasonic image data in a B (Brightness) mode that is an image mode, an ultrasonic diagnostic imaging system has been known that determines the number of spatially compounded visual directions, which serve as image parameters, in conformity with an image depth (display depth) of an ultrasonic image (Japanese Patent No. 4694692).

The B mode is an image mode for generating and displaying ultrasonic image data (B mode image data) where the amplitude of the reception signal according to reflected ultrasonic waves of ultrasonic waves transmitted to a subject is converted into the luminance The display depth is the depth of the deepest position in an ultrasonic image to be displayed. In the case of the B mode, the display depth is adjusted so as to accommodate an object to be observed within a display region. Accordingly, it is appropriate to set the number of spatially compounded visual directions in conformity with the display depth.

However, in a case where the image mode is a color Doppler mode or an elastography mode, an ROI (Region Of Interest) is arranged at a part of the display region and observation is made. Accordingly, setting of image parameters in conformity with the display depth is sometimes inappropriate. The color Doppler mode is an image mode for generating and displaying ultrasonic image data (C (color Doppler) mode image data) that represents the movement of a bloodstream or the like of a subject in colors. The elastography mode is a mode for displaying an image that represents the stiffness of tissue of the subject in colors in a manner overlaid on a B mode image, and for displaying a numerical value representing the stiffness of the tissue of the subject together with the B mode image. The C mode image data, or an image data that represents, in colors, the stiffness in the elastography mode, is displayed in the ROI on the B mode image. Image data on a measurement target that is the stiffness in the elastography mode is also displayed in the ROI.

Accordingly, there is a demand to set image parameters, in the color Doppler mode or the elastography mode, in conformity with the depth corresponding to the ROI.

### SUMMARY

The present invention has an object to apply appropriate image parameters in conformity with the depth of an ROI in an ultrasonic image.

To achieve at least one of the abovementioned objects, according to a first aspect of the present invention, an ultrasonic diagnostic apparatus reflecting one aspect of the present invention comprises:

a first hardware processor that generates ultrasonic image data, based on a reception signal obtained by an ultrasonic probe that transmits and receives an ultrasonic wave;

a storage that stores first image parameters associated with a depth corresponding to an ROI; and

a second hardware processor that causes the first hardware processor to generate the ultrasonic image data, based on the first image parameters.

According to a second aspect of the present invention, an ultrasonic diagnostic apparatus reflecting one aspect of the present invention comprises:

a first hardware processor that generates ultrasonic image data, based on a reception signal obtained by an ultrasonic probe that transmits and receives an ultrasonic wave to and from a subject;

a storage that stores first image parameters associated with a depth; and

a second hardware processor that causes the first hardware processor to generate the ultrasonic image data, based on the first image parameters, and registers default image parameters,

wherein when application of the first image parameters is turned off and the default image parameters are registered, the second hardware processor causes the first hardware processor to generate the ultrasonic image data, based on the default image parameters.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is an appearance diagram of an ultrasonic diagnostic apparatus of an embodiment of the present invention;

FIG. 2 is a block diagram showing a functional configuration of the ultrasonic diagnostic apparatus;

FIG. 3 is a block diagram showing a functional configuration of a C mode image generator;

FIG. 4 shows an input and output relationship of an MTI filter;

FIG. 5 shows C mode image parameter set groups;

FIG. 6 shows a B mode image parameter set group;

FIG. 7 shows a display depth, an ROI depth, and a focus depth;

FIG. 8 is a flowchart showing an image parameter set creation process;

FIG. 9A shows an image parameter set creation preparation screen;

FIG. 9B shows an image parameter set creation screen;

FIG. 10 shows an image parameter copy window;

FIG. 11A shows a total shallow copy of all image parameters, and an image parameter set subjected to a total deep copy;

FIG. 11B shows an image parameter set subjected to one-step shallower and one-step deeper copy of the total image parameter;

FIG. 12 shows an image parameter set subjected to total shallow copies and total deep copies of the repetitive number of color Doppler transmissions;

FIG. 13 is a flowchart showing a default image parameter set setting process;

FIG. 14A shows an image parameter set setting screen;

FIG. 14B shows an image parameter set selection window;

FIG. 15 is a flowchart showing a C mode image parameter set application process; and

FIG. 16 is a flowchart showing a C mode image parameter set application process subsequent to FIG. 15.

### DETAILED DESCRIPTION OF EMBODIMENTS

Hereinafter, one or more embodiments of the present invention will be described with reference to the drawings. However, the scope of the invention is not limited to the disclosed embodiments.

Referring to the accompanying drawings, embodiments according to the present invention are described.

### Embodiments

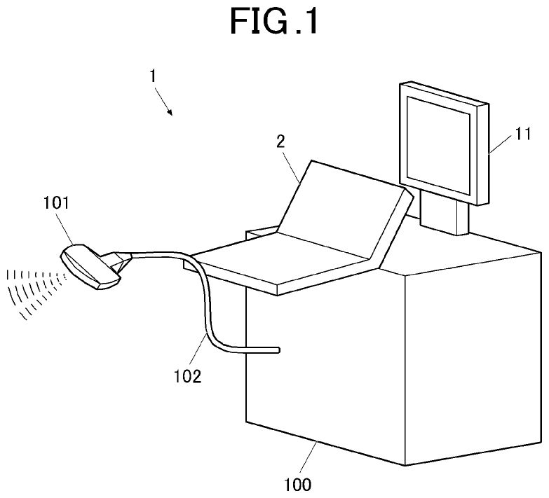

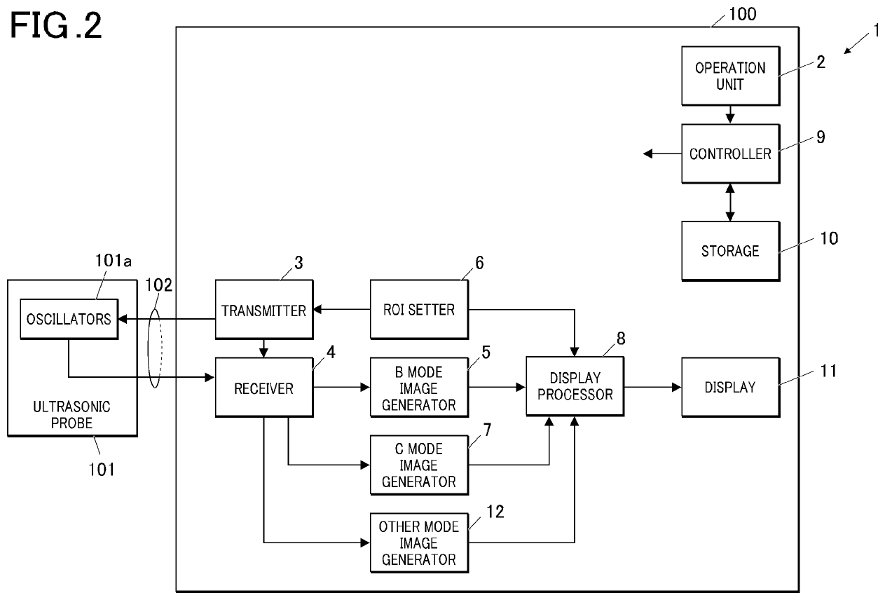

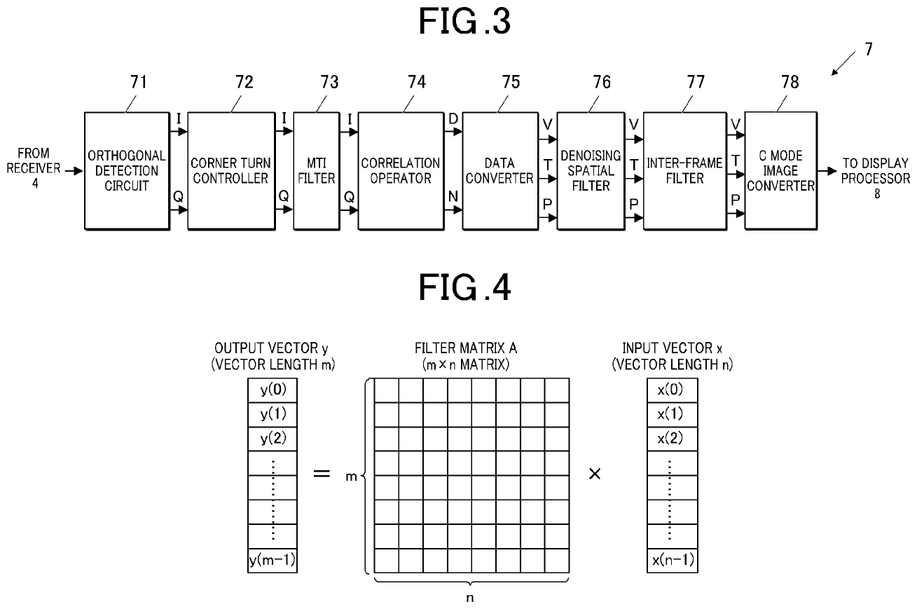

First, referring to FIGS. 1 to 3, a configuration of an apparatus of this embodiment is described. FIG. 1 is an appearance diagram of an ultrasonic diagnostic apparatus 1 of this embodiment. FIG. 2 is a block diagram showing a functional configuration of the ultrasonic diagnostic apparatus 1. FIG. 3 is a block diagram showing a functional configuration of a C mode image generator 7.

As shown in FIGS. 1 and 2, the ultrasonic diagnostic apparatus 1 of this embodiment is an apparatus that is installed in a medical institution, such as a hospital, and generates ultrasonic images of a subject, such as a living body of a patient, to be measured. This apparatus includes an ultrasonic diagnostic apparatus main body 100, and an ultrasonic probe 101. The ultrasonic probe 101 transmits ultrasonic waves (transmission ultrasonic waves) to a subject, such as a living body, not shown, and receives ultrasonic waves (reflected ultrasonic waves) reflected by the subject. The ultrasonic diagnostic apparatus main body 100 is connected to the ultrasonic probe 101 via a cable 102, transmits a drive signal that is an electric signal to the ultrasonic probe 101 to thereby cause the ultrasonic probe 101 to transmit transmission ultrasonic waves to the subject, and images the inner state of the subject as an ultrasonic image according to reflected ultrasonic waves from the inside of the subject received by the ultrasonic probe 101 on the basis of a reception signal that is an electric signal generated by the ultrasonic probe 101.

The ultrasonic probe 101 includes oscillators 101a made up of piezoelectric elements. The oscillators 101a are arranged, for example, in a one-dimensional array manner in a bearing direction (scanning direction). In this embodiment, for example, the ultrasonic probe 101 including 192 oscillators is adopted. The oscillator may be arranged in a two-dimensional manner. The number of oscillators 101a may be appropriately configured. In this embodiment, a linear scanning electronic scanning probe is adopted as the ultrasonic probe 101. However, any of an electronic scanning scheme and a mechanical scanning scheme may be adopted. Furthermore, any of a linear scanning scheme, a sector scanning scheme, and a convex scanning scheme may be adopted. The ultrasonic probe 101 may be replaced with another type.

The ultrasonic diagnostic apparatus main body 100 includes: an operation unit 2 serving as first to fourth operation units; a transmitter 3; a receiver 4; a B (Brightness) mode image generator 5; an ROI (Region Of Interest) setter 6; a C mode image generator 7; a display processor 8; a controller 9 serving as a creator, a setter and a registerer; a storage 10; a display 11, and an other mode image generator 12. The transmitter 3, the receiver 4, the B mode image generator 5, the ROI setter 6, the C mode image generator 7, the display processor 8, and the other mode image generator 12 function as an image generator.

The oscillators 101a each convert a drive signal (transmission electric signal) from the transmitter 3 into ultrasonic waves to generate an ultrasonic beam. Consequently, an operator arranges the ultrasonic probe 101 on the surface of the subject, thereby allowing the ultrasonic beam to be emitted to the inside of the subject. The ultrasonic probe 101 then receives reflected ultrasonic waves from the inside of the subject, and the oscillators 101a convert the reflected ultrasonic waves into a received electric signal, which is supplied to the receiver 4. Based on control by the controller 9, the transmitter 3 selects the oscillators 101a used by the ultrasonic probe 101, and individually changes timing at which voltages are supplied to the oscillators 101a, and the values of the voltages, thereby allowing control of the application position and emission direction of the ultrasonic beam transmitted by the ultrasonic probe 101.

The ultrasonic probe 101 may have parts of functions of the transmitter 3 and the receiver 4. For example, a configuration may be adopted where the ultrasonic probe 101 generates a drive signal in this ultrasonic probe 101 on the basis of a control signal (hereinafter called “transmission control signal”) for generating the drive signal output from the transmitter 3, the oscillators 101a convert the drive signal into ultrasonic waves and convert received reflected ultrasonic waves into a received electric signal, and based on the received electric signal, an after-mentioned reception signal is generated in the ultrasonic probe 101.

Furthermore, the ultrasonic probe 101 typically has the configuration of being electrically connected to the ultrasonic diagnostic apparatus main body 100 through the cable 102. However, the configuration is not limited thereto. For example, the ultrasonic probe 101 may have a configuration where a transmission signal and a reception signal are transmitted and received to and from the ultrasonic diagnostic apparatus main body 100 via wireless communication, such as of UWB (Ultra Wide Band) or the like. It is a matter of course that according to such a configuration, a configuration is necessarily adopted where the ultrasonic diagnostic apparatus main body 100 and the ultrasonic probe 101 include a wirelessly communicable communicator.

The operation unit 2 receives an input from the operator, and outputs an instruction based on the input by the operator to the controller 9. The operation unit 2 has a function of allowing the operator to select one of the B mode for displaying only a B mode image, an image mode (hereinafter called “C mode”) for displaying a C mode (a color Doppler mode or a color flow mode) image on the B mode image in an overlaid manner, and other image modes that include an image mode (hereinafter called “elastography mode”) for displaying an elastography image on the B mode image in an overlaid manner, an M (Motion) mode, a pulse wave Doppler mode, and a continuous wave Doppler mode. The operation unit 2 also has a function of accepting an input of designating the ROI position where the operator displays the C mode image or the elastography image on the B mode image. It is assumed that C mode images to be displayed further encompass C mode images in image modes that include a V (velocity) mode for color-displaying the flow velocity and direction of a bloodstream based on a bloodstream velocity V serving as a bloodstream signal indicating the state of the bloodstream, a P (power) mode for color-displaying the power of the bloodstream based on the power P of the bloodstream serving as a bloodstream signal, and a V-T mode for color-displaying the flow velocity and variance of the bloodstream based on the bloodstream velocity V and the variance T serving as a bloodstream signal. It is assumed that upon acceptance of a C mode input from the operator, the operation unit 2 further accepts an input of the display mode therefor. The display mode for the C mode image may include a T (variance) mode, and a dP (directed power) mode. As described above, the C mode (color Doppler mode) includes the V mode, the P mode, the T mode and the dP mode, and an image mode where at least two of them are combined.

It is assumed that the operation unit 2 is provided on a display screen of the display 11, and includes a touch panel for accepting a touch input by the operator.

The transmitter 3 generates at least the drive signal, and performs a transmission process of causing the ultrasonic probe 101 to transmit an ultrasonic beam. For example, the transmitter 3 performs the transmission process of generating the drive signal for transmitting an ultrasonic beam from the ultrasonic probe 101 including the oscillators 101a, and supplies the ultrasonic probe 101 with the drive signal that has a high voltage and occurs at predetermined timing, on the basis of the drive signal, thereby driving the oscillators 101a of the ultrasonic probe 101. Accordingly, the ultrasonic probe 101 converts the drive signal into ultrasonic waves, thereby allowing the ultrasonic beam to be emitted to the subject.

If the C mode is on, the transmitter 3 performs not only a transmission process for displaying the B mode image but also a transmission process for displaying the C mode image, according to the control of the controller 9.

For example, after an electric drive signal for displaying the B mode image is supplied, a drive signal for displaying the C mode image is supplied in the same direction (same line) n (n is more than ten and less than twenty, for example, 16) times, in all bearing (scanning) directions (all lines) in an ROI set by the ROI setter 6. In the transmission process, the transmitter 3 designates additional information to the transmission process for an image in the designated image mode, and supplies the additional information to the receiver 4.

The receiver 4 performs a reception process of generating a reception signal that is an electric RF (Radio Frequency) signal based on reflected ultrasonic waves, according to the control of the controller 9. The receiver 4 receives the reflected ultrasonic waves through the ultrasonic probe 101, for example, amplifies a received electric signal converted based on the reflected ultrasonic waves, and applies A/D conversion and phasing addition to the amplified signal, thereby generating a reception signal (sound ray data).

The receiver 4 obtains the additional information from the transmitter 3, and supplies the reception signal to the B mode image generator 5 if the obtained additional information is additional information for the B mode image, supplies the reception signal to the C mode image generator 7 if the obtained additional information is additional information for the C mode image, and supplies the reception signal to the other mode image generator 12 if the obtained additional information is additional information for any of the other image modes. Hereinafter, the reception signal for generating the B mode image is called “B mode reception signal,” the reception signal for generating the C mode image is called “C mode reception signal,” and the reception signal for generating the elastography image is called “elastography reception signal.” This analogously applies to the other image modes.

This embodiment may adopt the configuration where the receiver 4 selects whether the reception signal pertaining to a generated image frame is the B mode image or the C mode image, and supplies the selected signal to each block. However, the configuration is not limited thereto. For example, a configuration may be adopted that inputs the reception signal pertaining to the generated image frame into the B mode image generator 5, the C mode image generator 7 and the other mode image generator 12, thus achieving selection.

According to the control of the controller 9, the B mode image generator 5 applies envelope detection, logarithmic compression and the like to the B mode reception signal input from the receiver 4, adjusts the dynamic range and the gain, and achieves luminance conversion, thereby generating B mode image data and outputting the data to the display processor 8.

The ROI setter 6 sets an ROI of the C mode image in the transmitter 3 and the display processor 8, in response to ROI designation information input from the operator through the operation unit 2, according to the control of the controller 9.

According to the control of the controller 9, the C mode image generator 7 generates the C mode image data in response to the C mode reception signal input from the receiver 4, and outputs the data to the display processor 8. Here, referring to FIG. 3, the internal configuration of the C mode image generator 7 is described. As shown in FIG. 3, the C mode image generator 7 includes an orthogonal detection circuit 71, a corner turn controller 72, an MTI filter 73, a correlation operator 74, a data converter 75, a denoising spatial filter 76, an inter-frame filter 77, and a C mode image converter 78.

According to the control of the controller 9, the orthogonal detection circuit 71 orthogonally detects the C mode reception signal input from the receiver 4 to thereby calculate the phase difference between the obtained C mode reception signal and a reference signal, and obtains (complex) Doppler signals I and Q.

According to the control of the controller 9, the corner turn controller 72 arranges the Doppler signals I and Q input from the orthogonal detection circuit 71 on each identical acoustic line (line) in the depth direction from the ultrasonic probe 101 to the subject and in the ensemble direction of the repetitive number n of transmissions and receptions of ultrasonic waves, stores the signals in a memory (not shown), and reads the Doppler signals I and Q on a depth-by-depth basis in the ensemble direction.

The reception signals (Doppler signals I and Q) include not only a signal component of a bloodstream required to generate the C mode image, but also unnecessary information (clutter component) on blood vessel walls and tissue in a mixed manner

Here, referring to FIG. 4, the MTI filter 73 is described. FIG. 4 shows the input and output relationship of the MTI filter. According to the control of the controller 9, the MTI filter 73 filters the Doppler signals I and Q input from the corner turn controller 72 to remove the clutter component.

The complex number of the repetitive number n (packet size n) made up of the Doppler signals I and Q output from the corner turn controller 72 is assumed as packet data Sp. The packet data Sp is represented as n input data items x(0), x(1), . . . , x(n-1), x(0), x(1), . . . , x(n-1) are assumed to be arranged in a chronological order of generation or its reversed order.

As shown in FIG. 4, there is no mutual modulation between the clutter signal and the bloodstream signal.

The input and output of the MTI filter, which is a linear filter, is represented by the following equation (1).

y=Ax (1)

where y: an output vector indicating packet data items y(0), y(1), . . . , y(m-1) output from the MTI filter; A: an MTI filter coefficient matrix (filter matrix) (mxn); and x: an input vector indicating input data items x(0), x(1), . . . , x(n-1).

The MTI filter 73 is according to any of a scheme through a high-pass filter configured as an FIR (Finite Impulse Response) filter or an IIR (Infinite Impulse Response) filter, a scheme through a regression filter, a scheme through a principal component analysis, and a scheme through any combination thereof.

For example, the FIR filter coefficient matrix A_{FIR} serving as the MTI filter coefficient matrix A of the FIR filter is represented by the following equation (2).

$$\begin{matrix}[ Expression 1 ] & \\ {A}_{FIR} = [ \begin{matrix}{c}_{0} & {c}_{1} & … & {c}_{k - 1} & & & \\ & {c}_{0} & {c}_{1} & … & {c}_{k - 1} & & \\ & & … & … & … & … & \\ & & & {c}_{0} & {c}_{1} & … & {c}_{k - 1}\end{matrix} ] & ( 2 )\end{matrix}$$

where cᵢ: the FIR filter coefficient.

A polynomial regression filter coefficient matrix A_{reg} serving as the MTI filter coefficient matrix A of the polynomial regression filter is represented by the following equation (3).

### [Expression 2]

A_{reg}=I−Σᵢwᵢ(bᵢ·bᵢ^{T}) (3)

where i: the degree; wᵢ: the weight of the degree i, bi: the base vector with the degree i; and bᵢ^{T}: the Hermite transposed matrix of the base vector Bᵢ with the degree i.

Combination of the regression filter and the FIR filter can be achieved by arranging the filters in series. Preceding examples of this in-series scheme include U.S. Pat. No. 9,877,701, which describes a combination of a linear regression filter and an FIR filter. However, according to this in-series configuration, the regression filter and the FIR filter are required to be embedded. Accordingly, the circuit size (in a case of hardware) and the amount of calculation (in a case of software) become large.

To solve this, the MTI filter 73 may have a configuration where filters that are the regression filter and the FIR filter are represented as matrices, the MTI filter coefficient matrix A that is the product of the matrices is preliminarily calculated, and the packet data Sp is multiplied by the MTI filter coefficient matrix A. Accordingly, preliminary calculation of the matrix obtained by multiplication allows the MTI filter 73 to be configured only by a circuit or calculation of multiplying an input data string by the matrix.

Likewise, the scheme through the principal component analysis can represent the filter by a matrix. In the case of the scheme through the principal component analysis, the filter coefficients are calculated adaptively according to the input data. The MTI filter coefficient matrix A can be calculated by combining the calculated filter according to the scheme through the principal component analysis with the preliminarily calculated filter according to another scheme.

Returning to FIG. 3, according to the control of the controller 9, the correlation operator 74 calculates the real part D and the imaginary part N of the average S (the average of the phase difference vectors) of the autocorrelation operation of the Doppler signal according to the following equation (4), from the Doppler signals I and Q (complex Doppler signal z) obtained by filtering through the MTI filter 73.

$$\begin{matrix}[ Expression 3 ] & \\ S = {\sum }_{k = 0}^{n - 1} {z}_{k}^{*} \cdot {z}_{k + 1} = D + jN & ( 4 )\end{matrix}$$

According to the control of the controller 9, the data converter 75 calculates the bloodstream velocity V, the power P and the variance T, from the Doppler signals I and Q obtained by filtering through the MTI filter 73, and the real part D and the imaginary part N of the average S of the autocorrelation operation of the Doppler signal. More specifically, the data converter 75 calculates the bloodstream velocity V from the real part D and the imaginary part N of the average S of the autocorrelation operation of the Doppler signal, according to the following equation (5).

$$\begin{matrix}[ Expression 4 ] & \\ V = {tan}^{- 1} \frac{N}{D} & ( 5 )\end{matrix}$$

The data converter 75 calculates the power P that is the average of the intensities of the Doppler signals, from the Doppler signals I and Q (complex Doppler signal z), according to the following equation (6).

$$\begin{matrix}[ Expression 5 ] & \\ P = \frac{1}{n} {\sum }_{k = 0}^{n - 1} {\vert {z}_{k} \vert}^{2} & ( 6 )\end{matrix}$$

The data converter 75 calculates the variance T that is the ratio between the magnitude of the phase difference vector and the power (note that the value is inverted by subtraction from one), from the real part D and the imaginary part N of the average S of the autocorrelation operation of the Doppler signal, according to the following equation (7).

$$\begin{matrix}[ Expression 6 ] & \\ T = 1 - \frac{\sqrt{{D}^{2} + {N}^{2}}}{P} & ( 7 )\end{matrix}$$

The denoising spatial filter 76 filters the power P, the bloodstream velocity V and the variance T calculated by the data converter 75. The denoising spatial filter 76 includes a keyhole filter and a spatial filter (neither shown).

The keyhole filter filters the power P constituting the frame of the C mode image, the bloodstream velocity V and the variance T, and removes noise. In the V mode and the V-T mode, the keyhole filter filters the bloodstream velocity V by removing the bloodstream velocity V in a region that is for removal and is set by the bloodstream velocity V and the power P calculated by the data converter 75. In the V mode and the V-T mode, the bloodstream velocity V is used for image display (coloring). In the P mode, the keyhole filter filters the power P by removing the power P in a region that is for removal and is set by the bloodstream velocity V and the power P calculated by the data converter 75. In the P mode, the power P is used for image display (coloring).

More specifically, in the V mode and the V-T mode, the keyhole filter regards, as clutter noise, the bloodstream signal in a region where the bloodstream velocity V is smaller than a predetermined threshold, and regards, as background noise, the bloodstream signal in a region where the power P is lower than a predetermined threshold, and removes the bloodstream velocity V in these regions. In the P mode, the keyhole filter regards, as clutter noise, the bloodstream signal in a region where the bloodstream velocity V is smaller than a predetermined threshold, and regards, as background noise, the bloodstream signal in a region where the power P is lower than a predetermined threshold, and removes the power P in these regions.

The spatial filter is a two-dimensional weighted average filter for smoothing data on the bloodstream velocity V, the power P and the variance T, which constitute the frame of the C mode image. In the V mode or the V-T mode, the spatial filter filters the bloodstream velocity V filtered by the keyhole filter, and the variance T calculated by the data converter 75. In the P mode, the spatial filter filters the power P filtered by the keyhole filter.

The inter-frame filter 77 filters the bloodstream component of each of frames constituting the C mode image in conformity with the display mode where an operation input is made through the operation unit 2, from among the bloodstream velocity V, the power P and the variance T filtered through the denoising spatial filter 76, so as to smooth variation between frames and leave afterimages.

The C mode image converter 78 color-maps the bloodstream velocity V, the power P and the variance T filtered through the inter-frame filter 77, and converts them into ROI C mode image data, thus generating the data.

Returning to FIG. 2, according to the control of the controller 9, the other mode image generator 12 generates image data (note that image data on an elastography image having the size of the ROI in the elastography mode) in another image mode (the elastography mode, the M mode, the pulse wave Doppler mode, the continuous wave Doppler mode or the like), from the reception signal in the other image mode input through the receiver 4, and outputs the data to the display processor 8.

According to the control of the controller 9, the display processor 8 performs a process of constructing display image data to be displayed on the display 11 and causing the display 11 to display the display image data. In particular, if the B mode is selected, a process of including the B mode image in the B mode image data generated by the B mode image generator 5, as an ultrasonic image, in the display image data, is performed. If the C mode is selected, a process of generating, as an ultrasonic image, combined image data where the C mode image in the C mode image data generated by the C mode image generator 7 is overlaid at the position of the ROI selected on the B mode image generated by the B mode image generator 5, and of including the data in the display image data. If the elastography mode is selected, a process of generating, as an ultrasonic image, combined image data where the elastography image in the elastography image data generated by the other mode image generator 12 is overlaid at the position of the ROI selected on the B mode image generated by the B mode image generator 5, and of including the data in the display image data. If the M mode, the pulse wave Doppler mode or the continuous wave Doppler mode is selected, a process of generating, as an ultrasonic image, combined image data where the B mode image generated by the B mode image generator 5 and the image in the image data in the other image mode are to be displayed in parallel, and of including the data in the display image data.

According to the control of the controller 9, the display processor 8 appropriately applies an image processing in conformity with the image parameters of each applied image mode, to the ultrasonic image data in the corresponding image mode.

The controller 9 includes, for example, a CPU (Central Processing Unit), a ROM (Read Only Memory) and a RAM (Random Access Memory), reads various process programs, such as system programs, stored in the ROM, deploys the programs on the RAM, and controls the operation of each component of the ultrasonic diagnostic apparatus 1 according to the deployed programs. The RAM forms a work area that temporarily stores the various programs to be executed by the CPU and data related to these programs. The ROM is made up of a nonvolatile memory, such as of a semiconductor, and stores the system program for the ultrasonic diagnostic apparatus 1, the various process programs executable on the system program, and various data items. These programs are stored in a form of computer-readable program code. The CPU sequentially executes the operations according to the program code. The ROM stores, for example, an image parameter set creation program, a default image parameter set setting program, and a C mode image parameter set application program, which are for respectively performing the image parameter set creation process, the default image parameter set setting process, and the C mode image parameter set application process, described later.

The storage 10 is made up of, for example, a large capacity recording medium, such as an HDD (Hard Disk Drive), and stores the ultrasonic image data (the B mode image data, the C mode image data, and the combined image data) and image parameter set groups 200, 300 and 400, described later.

The display 11 is what is called a monitor, such as an LCD (Liquid Crystal Display), or an EL (ElectroLuminescence) display, for displaying the image data output from the display processor 8.

As for each component included in the ultrasonic diagnostic apparatus 1, a part of or the entire function of each functional block can be achieved as a hardware circuit, such as an integrated circuit. The integrated circuit is, for example, an LSI (Large Scale Integration). The LSI is called an IC (Integrated Circuit), a system LSI, a super LSI, or an ultra LSI with respect to the difference in degree of integration, in some cases. Achievement of an integrated circuit is not limited to that of LSI. Alternatively, achievement may be made by a dedicated circuit or a general purpose processor. Further alternatively, an FPGA (Field Programmable Gate Array), or a reconfigurable processor, which allows connection and configuration of circuit cells in an LSI, can be reconfigured may be adopted. A part of or the entire function of each functional block may be executed by software. In this case, the software is stored in one or more storage media, such as ROMs, an optical disk, or a hard disk. This software is executed by an operation processor.

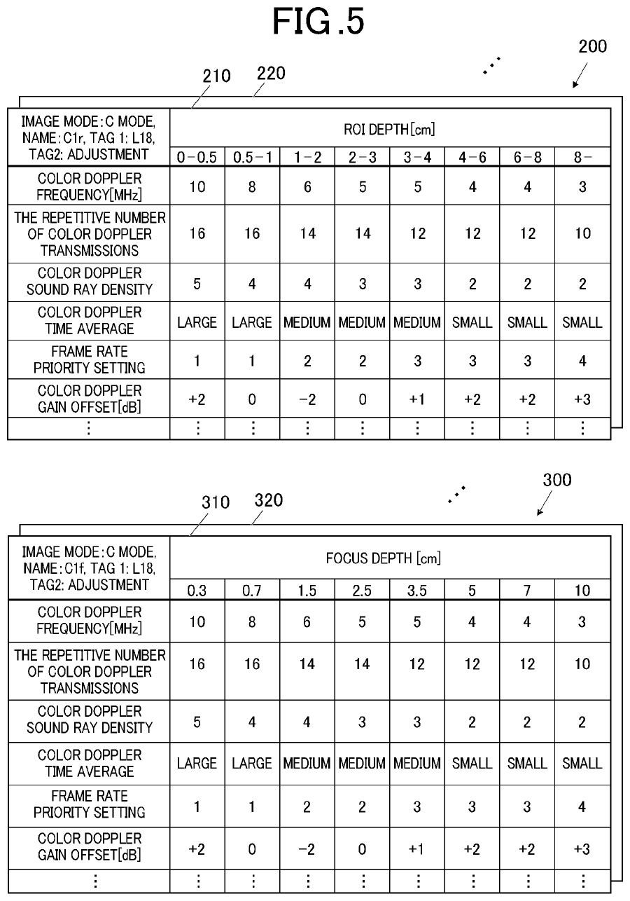

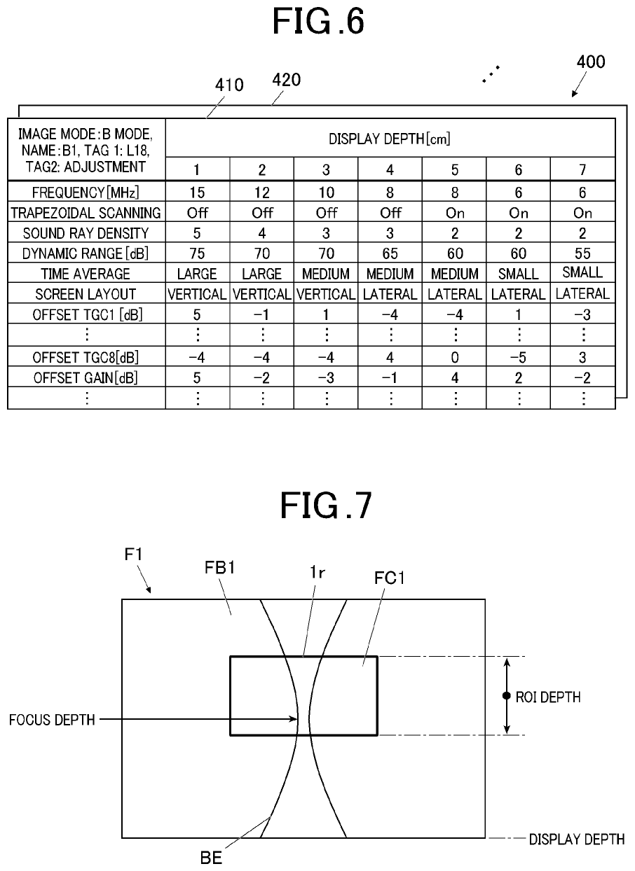

Next, referring to FIGS. 5 to 7, the configurations of the image parameter set groups 200, 300 and 400 stored in the storage 10 are described. FIG. 5 shows the C mode image parameter set groups 200 and 300. FIG. 6 shows the B mode image parameter set group 400. FIG. 7 shows the display depth, ROI depth, and focus depth.

The image parameter set group 200 shown in FIG. 5 is a C mode image parameter set group, and is a table for storing image parameter sets, each of which is a combination of setting values of image parameters in association with each of the ROI depths. The image parameter set group 300 shown in FIG. 5 is a C mode image parameter set group, and is a table for storing image parameter sets, each of which is a combination of setting values of image parameters in association with each of the focus depths. The image parameter set group 400 shown in FIG. 6 is a B mode image parameter set group, and is a table for storing image parameter sets, each of which is a combination of setting values of image parameters in association with each of the display depths.

Here, referring to FIG. 7, the display depth, the ROI depth and the focus depth are described. A C mode combined image F1 shown in FIG. 7 is an ultrasonic image where a C mode image FC1 in an ROI 1r is overlaid on a B mode image FB1 serving as a background image. The upper end of the B mode image FB1 corresponds to the body surface of a subject.

The display depth is the depth of the lower end of the entire display region of the ultrasonic image with reference to the body surface, and is, for example, the depth of the lower end of the B mode image FB1. The ROI depth is the depth of a position preset between the upper end and the lower end of the ROI with reference to the body surface. For example, provided that the distance between the upper end and the lower end of the ROI is 100%, the depth is a position at a predetermined ratio from the upper end (assumed as 50% in this embodiment (the center between the upper end and the lower end)). Note that the predetermined ratio may be other than 50%.

The focus depth is a depth corresponding to the focal point of an ultrasonic beam with reference to the body surface, and is the depth of the focal point of an ultrasonic beam BE of the C mode image FC1, for example. Note that the ultrasonic beam BE indicates one ultrasonic beam among transmissions for scanning the ROI. The focus depth may be configured to be determined according to the ROI depth (dynamically associated with the ROI depth), or configured to allow the operator to set the depth through the operation unit 2. It is assumed that in the former case, the C mode focus depth used as the image parameter is the focus depth coinciding with the ROI depth, or the focus depth closest to the ROI depth among the focus depths that the ultrasonic diagnostic apparatus 1 can take.

Although not shown, if the image mode is an image mode using a sample gate, such as the pulse wave Doppler mode, the position at the center of the line segment between the upper gate center and the lower gate center of the sample gate is used as the gate depth, for example.

The C mode image parameter set group 200 includes image parameter sets 210, 220, . . . provided with respect to the ultrasonic probe type, observation site, ultrasonic diagnosis field and the like.

The image parameter set 210 includes an image parameter set name, a tag 1, and a tag 2. The name, the tag 1, and the tag 2 function as identification information on the image parameter set 210. Accordingly, for example, a configuration may be adopted that assigns the same name to multiple image parameter sets, and assigns different tags to the image parameter sets. An example is described where at most two are registered for each image parameter set. However, the number is not limited thereto.

The name is the name of an image parameter set, and is assumed as the identification information (C1r etc.). However, the name is not limited thereto, and may be the observation site, for example. The tag may be the type of the ultrasonic probe 101, the ultrasonic diagnosis field or the like in the ultrasonic diagnosis using the image parameter set, and may be freely set by the operator. The name and the tag may be used for keyword search and tag search for the image parameter set.

The image parameter set 210 is assumed to have the color Doppler frequency, the number of color Doppler repetitive transmissions, the color Doppler sound ray density, the color Doppler time average, the frame rate priority setting, and the color Doppler gain offset setting value, as the C mode image parameters, in association with each of ROI depths (for example, “0 to 0.5 [cm] ” to “8 [cm]-”).

The color Doppler (transmission) frequency is the frequency [MHz] of the transmission ultrasonic waves during C mode image data generation. The number of color Doppler repetitive transmissions is the number of transmission ultrasonic waves repetitively transmitted to the same scanning position during generation of one frame of the C mode image data. The color Doppler sound ray density is information indicating the sound ray density of transmission ultrasonic waves emitted from the ultrasonic probe 101 during C mode image data generation. The setting value of the color Doppler sound ray density is, for example, the degree of the sound ray density as a numerical value (1 to 5). Reduction in color Doppler sound ray density increases the frame rate to reduce the image quality, while increase in this density reduces the frame rate to increase the image quality.

The color Doppler time average is an amount indicating the magnitude of a process of calculating the arithmetic average or a weighted average of C mode images (pixel values) of temporally sequential multiple frames among C mode images during C mode image data generation, and indicates the magnitude of the time average process in three levels, i.e., strong, medium and weak, for example. The stronger the magnitude is, the larger the number of frames used for calculating the arithmetic average is or the heavier the weight of previous frames is. The weaker the magnitude is, the smaller the number of frames used for calculating the arithmetic average is or the weaker the weight of the previous frames is. The time average is information indicating the magnitude of a process of overlaying multiple frames of images to remove noise and of intensifying the true signal (persistence).

The frame rate priority setting is information indicating the magnitude of a process of prioritizing (increasing) the frame rate rather than the image quality during C mode image data generation. The process of prioritizing the frame rate is a process of prioritizing the frame rate of a combined image by, for example, limiting the scanning parameters of the B mode image, which is to be combined with the C mode image. The scanning parameters limited in the B mode image are, for example, the B mode sound ray density, THI (Tissue Harmonic Imaging), multistage focus, and synthetic aperture.

Reduction in B mode sound ray density increases the frame rate to reduce the image quality, while increase in this density reduces the frame rate to increase the image quality. Accordingly, the stricter the limitation on the B mode sound ray density (limiting the sound ray density to a smaller degree), the more the frame rate increases.

The THI is THI according to a pulse inversion scheme. During B mode image data generation, this scheme outputs a drive signal twice from the transmitter with waveforms with the positive and negative being inverted, and adds together the reception signals obtained by the receiver to cancel basic wave components and obtain harmonic components, thereby improving the resolution of the ultrasonic image. THI is not only the THI according to the pulse inversion scheme, but also filter-THI that filters reception signals to remove components other than harmonic components and obtain the harmonic components, and achieves imaging According to this scheme, only one ultrasonic wave transmission and reception is required per acoustic line. THI limitation information is, for example, information for performing the THI process to reduce the frame rate, or for performing no THI process or performing the filter-THI to improve the frame rate.

The multistage focus is a method that transmits and receives ultrasonic waves multiple times with the transmission focus depth being varied, and combines reception signals obtained by the multiple-time transmission and reception to generate one acoustic line of the reception signal. The multistage focus requires multiple-time transmission and reception per acoustic line. Accordingly, the frame rate is reduced in comparison with a case without using the multistage focus. The multistage focus limitation information is information for performing the multistage focus process to reduce the frame rate or for performing no multistage focus process to increase the frame rate.

The synthetic aperture is a method that selects multiple reception apertures of oscillators 101a from among those of all the oscillators 101a of the ultrasonic probe 101, combines reception signals of two ultrasonic wave transmissions and receptions, and achieves an effect of receiving through a large aperture. This is adopted because an upper limit value of the number of oscillators 101a (channels) usable as reception apertures is predetermined. Note that to achieve a large aperture by two transmissions and receptions of ultrasonic waves, the frame rate is halved. The synthetic aperture limitation information is information for performing the synthetic aperture process to reduce the frame rate or for performing no synthetic aperture process to increase the frame rate.

The color Doppler gain offset (offset gain) is information indicating a gain value [dB] that is to be added as an offset to the C mode image. Provided that the color Doppler gain offset is enabled in a case where the gain is adjusted through a normal operation of increasing and reducing the gain value of the display C mode image by an operator's normal gain input, the normal gain adjustment value +the offset gain is added to each pixel value of the C mode image.

In the image parameter set 210, the setting value of the color Doppler frequency is set to be high for prioritizing the resolution if the ROI depth is small, and this value is set to be low for prioritizing the penetration sensitivity if the ROI depth is large. In the image parameter set 210, the setting value of the number of color Doppler repetitive transmissions is increased in order to improve the S/N (Signal-Noise Ratio) if the ROI depth is small, and this value is reduced in order to secure the frame rate if the ROI depth is large.

In the image parameter set 210, the setting value of the color Doppler sound ray density is increased in order to improve the image quality if the ROI depth is small, and this value is reduced in order to secure the frame rate if the ROI depth is large. In the image parameter set 210, the setting value of the color Doppler time average is increased because of a high frame rate if the ROI depth is small, and this value is reduced because of a low frame rate if the ROI depth is large. In the image parameter set 210, the setting value of the frame rate priority setting is reduced to prioritize the image quality (the degree of prioritizing the frame rate is reduced) if the ROI depth is small, and this value is increased (intensified) in order to secure the frame rate if the ROI depth is large.

In the image parameter set 210, the setting values of the image parameters that include the color Doppler frequency, the number of color Doppler repetitive transmissions, the color Doppler sound ray density, the color Doppler time average, the frame rate priority setting, and the color Doppler gain offset, are dynamically associated with the ROI depth.

Although not shown, the image parameter set 210 includes the setting values of other image parameters that are not associated with the ROI depth. The image parameters that are not dynamically associated with the ROI depth include, for example, the flow velocity scale, the steering angle, and the ROI size. A flow velocity scale of about 15 [cm/s] is typically appropriate for an abdomen (a liver or kidneys). However, in actual usage, the flow velocity scale is adjusted in conformity with the subject. Dynamical associated change in flow velocity scale in a case where the ROI position is adjusted after the above adjustment is undesirable. As for the steering angle, steering is typically performed to observe a carotid artery. In conformity with the blood vessel drawing angle, the steering angle is adjusted. Dynamical associated change in steering angle in a case where the ROI position is adjusted after the above adjustment is undesirable. The ROI size is adjusted in conformity with a target during actual usage. Unintentional change in ROI size when the ROI is subsequently moved is undesirable.

Even with the image parameters that are not dynamically associated with the depth, there is a case that is intended to be applied when the image parameter set dedicated to a specific diagnostic site is selected. For example, in the case of the flow velocity scale, about 15 [cm/s] is typically appropriate for an abdomen. However, since the bloodstream velocity is high in an aorta, it is appropriate to set the velocity to about 25 [cm/s]. In the case of the steering angle, among cervical blood vessels, steering is performed for a carotid artery but is not performed for a vertebral artery (or with a small steering angle). The image parameter set in the elastography mode includes the setting values of other image parameters that are not dynamically associated with the ROI depth (or the focus depth). The other image parameters that are not dynamically associated with the ROI depth include the ROI size.

The C mode image parameter set group 300 includes image parameter sets 310, 320, . . . provided with respect to the ultrasonic probe type, observation site, ultrasonic diagnosis field and the like.

The image parameter set 310 includes an image parameter set name, a tag 1, and a tag 2. The image parameter set 310 is assumed to have the color Doppler frequency, the number of color Doppler repetitive transmissions, the color Doppler sound ray density, the color Doppler time average, the frame rate priority setting, and the color Doppler gain offset setting value, as the C mode image parameters, in association with each of focus depths (for example, “0.3” to “10 [cm]”).

In the image parameter set 310, as with the image parameter set 210, the image parameters of the color Doppler frequency, the number of color Doppler repetitive transmissions, the color Doppler sound ray density, the color Doppler time average, the frame rate priority setting, and the color Doppler gain offset, are dynamically associated with the focus depth. Although not shown, the image parameter set 310 includes the setting values of other image parameters that are not associated with the focus depth. It is assumed that as the image parameter set for the C mode, the image parameter set group that includes the setting values of the image parameters associated with the display depth are also stored in the storage 10.

The B mode image parameter set group 400 shown in FIG. 6 includes image parameter sets 410, 420, . . . provided with respect to the ultrasonic probe type, observation site, ultrasonic diagnosis field and the like.

The image parameter set 410 includes an image parameter set name, a tag 1, and a tag 2. The image parameter set 410 stores setting values of the (transmission) frequency, the trapezoidal scanning, sound ray density, dynamic range, time average, screen layout, offset TGCs (Time Gain Compensations) 1 to 8, offset gain and the like, as B mode image parameters, in association with display depths (for example, 1 to 7 [cm] (intervals of 1 [cm])).

The (transmission) frequency is the frequency [MHz] of the transmission ultrasonic waves during B mode image data creation. The trapezoidal scanning is information indicating whether or not (on or off) to perform trapezoidal scanning for generating trapezoidal B mode image data by changing the angle of the sound ray of each oscillator using the linear-shaped ultrasonic probe 101. Likewise, also in a case of a convex-shaped ultrasonic probe 101, the range of scanning can be widened by changing the degree of the sound ray of each oscillator. This can also be called trapezoidal scanning for the sake of convenience. The sound ray density is information indicating the sound ray density during B mode image data generation.

The dynamic range is information indicating the dB value to be allocated to the luminance gradation of the image data to be generated in the luminance range of the sound ray data exceeding 100 [dB]. The time average is a time average during B mode image data generation. The screen layout is information indicating a display arrangement between a vertical display arrangement and a lateral display arrangement in a case where two-screen display is adopted.

The offset TGCs 1 to 8 are information indicating gain values (luminance value) [dB] to be added to eight regions, as offsets that are correction values for times corresponding to the distances of the respective regions, the regions being obtained by dividing the B mode image into eight regions in the depth direction. Here, for example, the regions for the TGCs through a normal operation and the regions for the offset TGCs are assumed to be the same. Note that the number of offset TGCs and the number of normal TGCs are not limited to eight. Provided that the offset TGC is effective when TGC through a normal operation of increasing or reducing the luminance value of each region of the display B mode image is performed by the operator's input, the luminance value of normal TGC +offset TGC is added to each pixel value in each corresponding region of the B mode image. The offset gain is an offset gain during B mode image data generation.

In the image parameter set 410, the setting value of frequency is set to a large value if the display depth is small, and the value is set to a small value if the display depth is large. In the image parameter set 410, the setting value of trapezoidal scanning is disabled (off) if the display depth is small, and the value is enabled (on) if the display depth is large. A configuration may be adopted where the setting value of trapezoidal scanning is a setting value for reducing the angle of the sound ray if the display depth is small, and the value is a setting value for increasing the angle of the sound ray if the display depth is large.

In the image parameter set 410, the setting value of the sound ray density is set to a large value if the display depth is small, and the value is set to a small value if the display depth is large. In the image parameter set 410, the setting value of the time average is set to a large value if the display depth is small, and the value is set to a small value if the display depth is large.

In the image parameter set 410, the setting value of the dynamic range is set to a large value if the display depth is small, and the value is set to a small value if the display depth is large. In the image parameter set 410, the setting value of the screen layout is set to a value indicating the vertical layout if the display depth is small, and the value is set to a value indicating the lateral layout if the display depth is large.

In the image parameter set 410, the trapezoidal scanning is enabled if the display depth is large. If the trapezoidal scanning is enabled, the image quality is degraded at a deep part because the sound ray intervals are large. Although not shown, to compensate it, the sound ray density may be increased at the deep part. However, increase in sound ray density reduces the frame rate and reduces the tracking performance. Accordingly, the time average is set to be small at the deep part. Since increase in depth and reduction in time average reduce the S/N, the dynamic range is set to be small. In conformity therewith, the gain (the offset TGC, and the offset gain) may be changed. As described above, it is configured such that in the image parameter set 410, pieces of setting information on multiple image parameters cooperate with each other, thereby allowing favorable ultrasonic image data to be obtained.

In the image parameter set 410, the setting values of the image parameters that are the frequency, trapezoidal scanning, sound ray density, dynamic range, time average, screen layout, offset TGC, and the offset gain are dynamically associated with the display depth. Although not shown, the image parameter set 410 includes the setting values of other image parameters that are not dynamically associated with the display depth.

In this embodiment, it is assumed that the image parameter set groups 200, 300 and 400 are stored in the storage 10. However, the assumption is not limited thereto. Alternatively, it may be assumed that image parameter set groups for other image modes, such as the elastography mode, the M mode, the pulse wave Doppler mode, and the continuous wave Doppler mode, are stored in the storage 10.

For example, the image parameter set for the pulse wave Doppler mode has setting values of image parameters corresponding to the gate depth or the focus depth. The image parameters include at least the transmission frequency. The image parameter set for the continuous wave Doppler mode has setting values of image parameters corresponding to the focus depth. The image parameters include at least the transmission frequency. The image parameter set for the M mode has setting values of image parameters corresponding to the display depth.

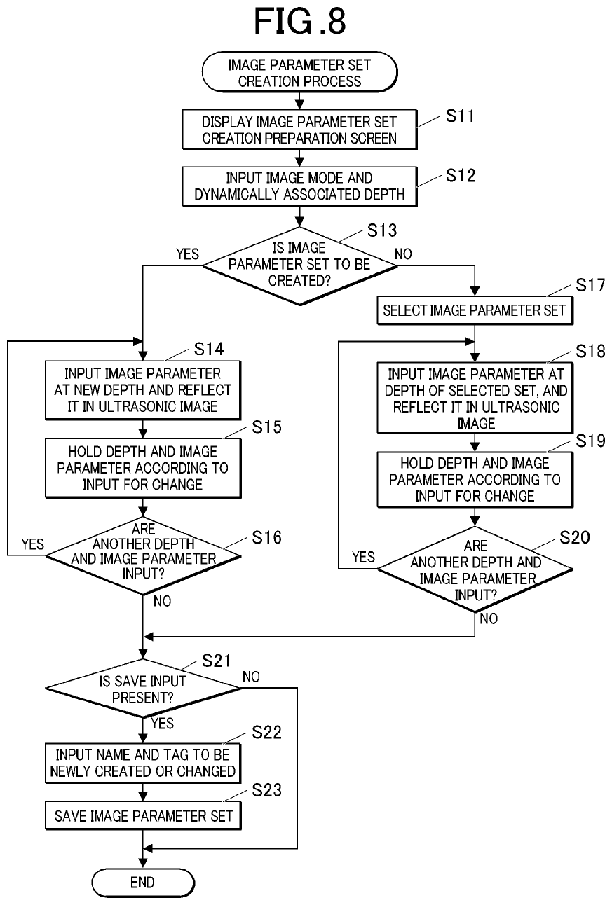

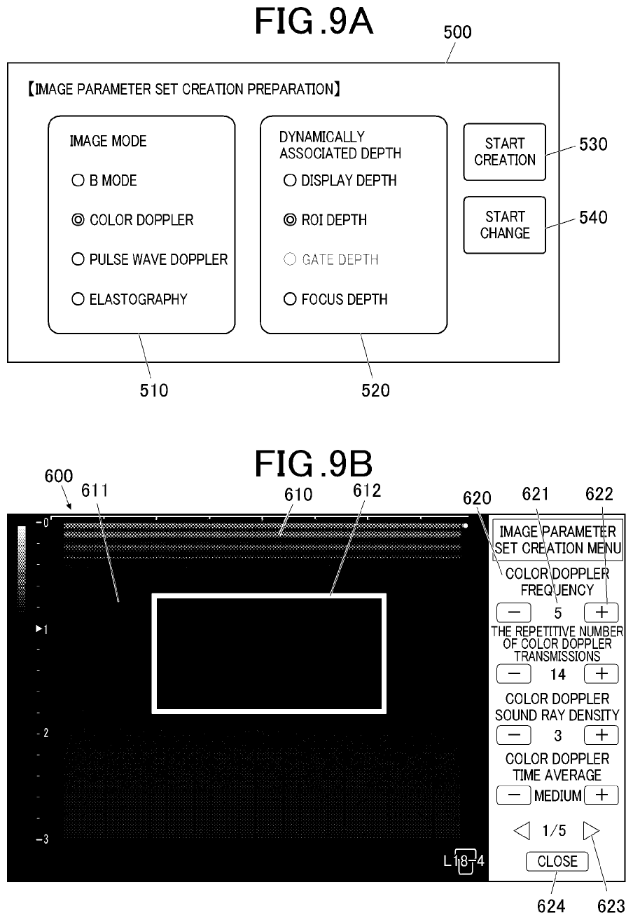

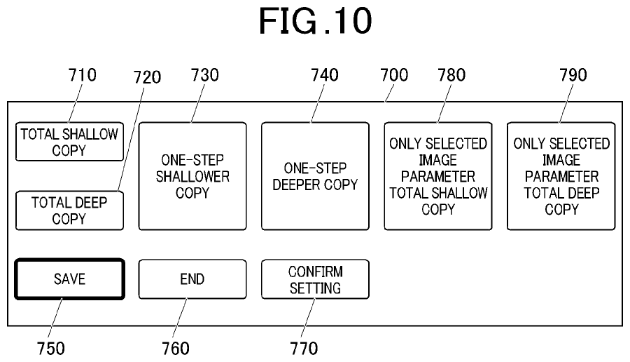

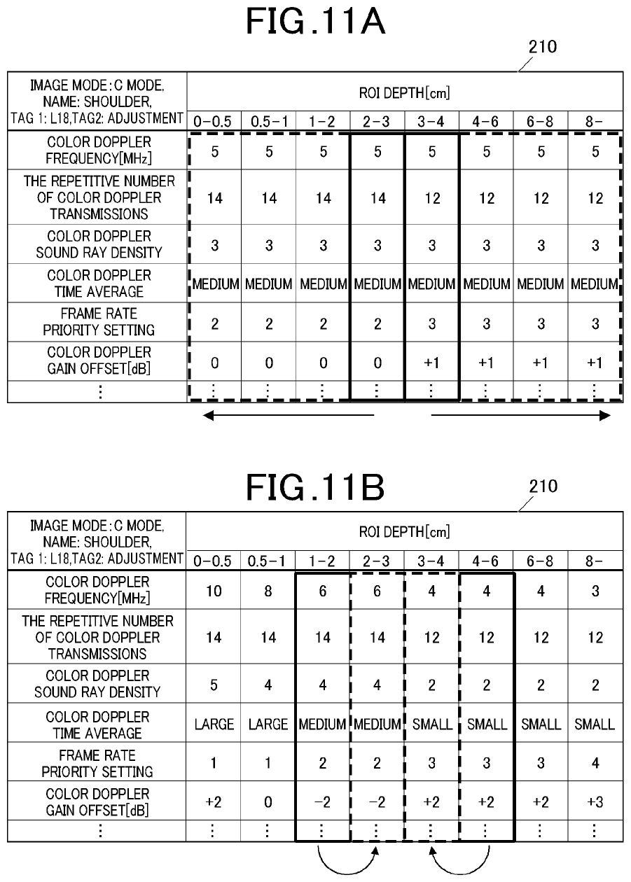

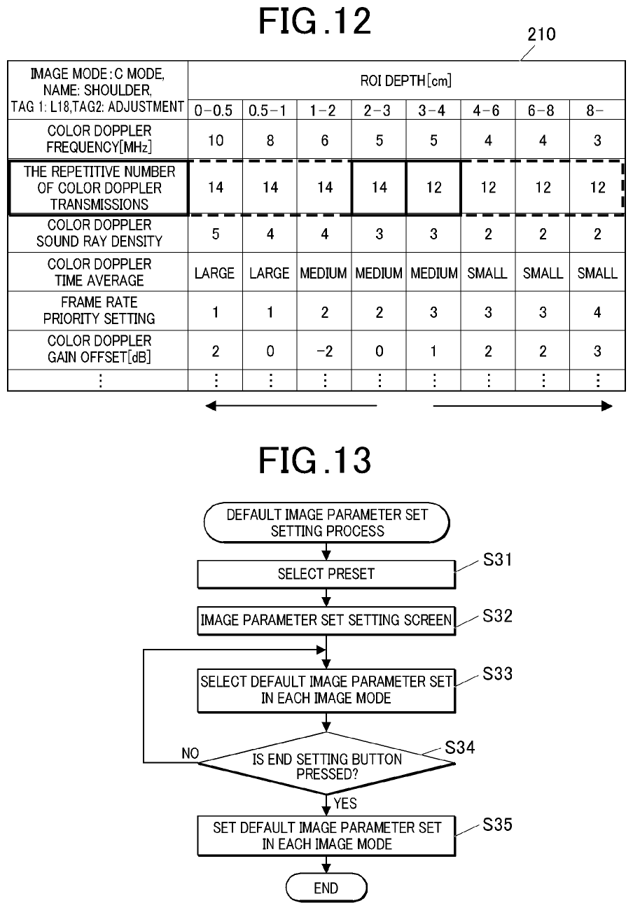

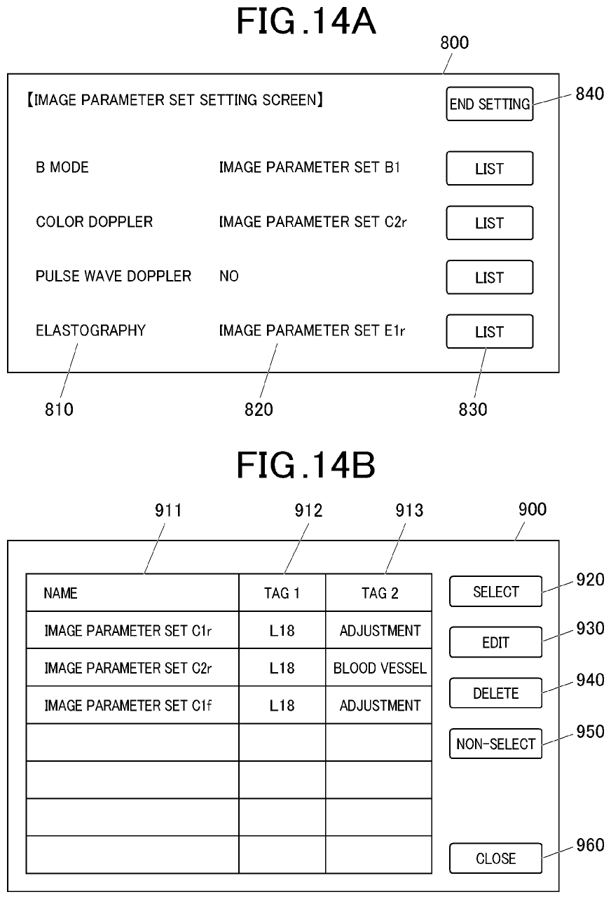

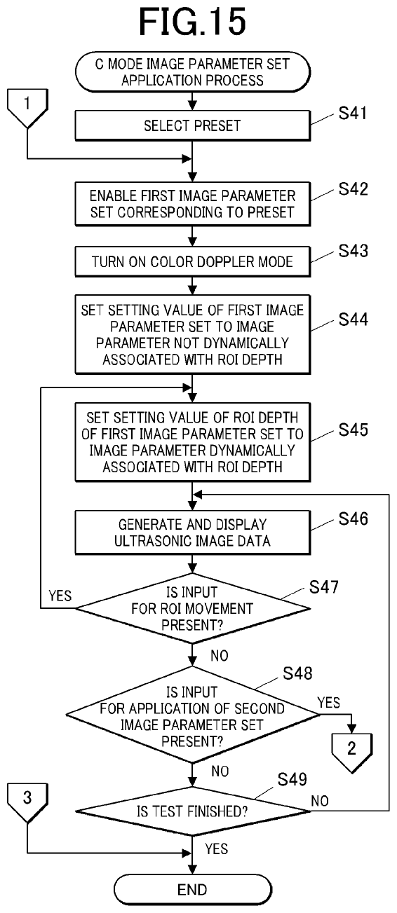

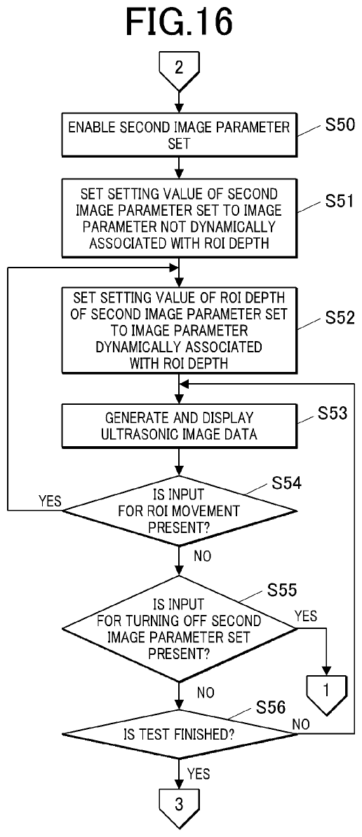

Next, referring to FIGS. 8 to 16, the operations of the ultrasonic diagnostic apparatus 1 are described. FIG. 8 is the flowchart showing an image parameter set creation process. FIG. 9A shows an image parameter set creation preparation screen 500. FIG. 9B shows an image parameter set creation screen 600. FIG. 10 shows an image parameter copy window 700. FIG. 11A shows a total shallow copy of all image parameters, and an image parameter set 210 subjected to a total deep copy. FIG. 11B shows an image parameter set 210 subjected to one-step shallower and one-step deeper copy. FIG. 12 shows an image parameter set 210 subjected to total shallow copies and total deep copies of the repetitive number of color Doppler transmissions. FIG. 13 is a flowchart showing a default image parameter set setting process. FIG. 14A shows an image parameter set setting screen 800. FIG. 14B shows an image parameter set selection window 900. FIG. 15 is a flowchart showing a C mode image parameter set application process. FIG. 16 is a flowchart showing a C mode image parameter set application process subsequent to FIG. 15.

First, referring to FIGS. 8 to 12, the image parameter set creation process executed by the ultrasonic diagnostic apparatus 1 is described. The image parameter set creation process is a process of newly creating or changing the image parameter set and storing the set while allowing an operator, such as a doctor or a technician, to confirming display content of the image parameter set on a live ultrasonic image (live image) in a desired image mode. It is assumed that a technician as a subject is preliminarily laid on a bed in a room provided with the ultrasonic diagnostic apparatus 1. Note that the image parameter set creation process may be performed in an apparatus designing stage.

In the ultrasonic diagnostic apparatus 1, for example, input of an execution instruction for the image parameter set creation process from the operator through the operation unit 2 serves as a trigger, and the controller 9 executes the image parameter set creation process according to the image parameter set creation program stored in the ROM.

As shown in FIG. 8, first, the controller 9 creates image parameter set creation preparation screen information, and displays the information on the display 11 (step S11). In step S11, for example, the image parameter set creation preparation screen 500 shown in FIG. 9A is displayed. The image parameter set creation preparation screen 500 includes an image mode selection region 510, a dynamically associated depth selection region 520, a creation start button 530, and a change start button 540.

The image mode selection region 510 is a selection region for selecting a desired image mode corresponding to the image parameter set to be created or changed, and includes radio buttons for the respective modes, for example. The dynamically associated depth selection region 520 is an input region for a desired dynamically associated depth (depth type) dynamically associated with the image mode selected through the image mode selection region 510, and includes radio buttons for the respective dynamically associated depths, for example.

The creation start button 530 is a button for accepting an input of an instruction of creating the image parameter set at the dynamically associated depth selected through the dynamically associated depth selection region 520 in the image mode selected through the image mode selection region 510. The change start button 540 is a button for accepting an input of an instruction for changing the image parameter set at the dynamically associated depth selected through the dynamically associated depth selection region 520 in the image mode selected through the image mode selection region 510.

The controller 9 then accepts an input of the image mode and the dynamically associated depth from the operator through the operation unit 2, in association with the image parameter set creation preparation screen displayed in step S11 (step S12).

A configuration may be adopted in step S11, the dynamically associated depth selection region is not provided for the image parameter set creation preparation screen, and the dynamically associated depth is predefined. In this case, in step S12, for example, if the image mode is the B mode, the display depth is selected as the dynamically associated depth, and if the image mode is the color Doppler mode or the elastography mode, the

ROI depth is selected as the dynamically associated depth, and if the image mode is the pulse wave Doppler mode, the gate depth is selected as the dynamically associated depth.

The controller 9 then accepts, from the operator through the operation unit 2, input through the creation start button 530 or the change start button 540, in association with the image parameter set creation preparation screen displayed in step S11, and determines whether to newly create (change) the image parameter set or not (step S13).

If the set is newly created (step S13; YES), the controller 9 displays, on the display 11, image parameter set creation screen information associated with the input value of the dynamically associated depth, accepts, through the operation unit 2, input of the value of the dynamically associated depth, input in step S12, of the image parameter set to be newly created, and the setting values of the dynamically associated depth value and the image parameters in the image mode input in step S12, and reflects input information (the value of the dynamically associated depth, and the setting value of the image parameter in the image mode), into the live ultrasonic image (live image) of the image parameter set creation screen information (step S14).

In step S14, for example, the image parameter set creation screen 600 shown in FIG. 9B is displayed. The image parameter set creation screen 600 includes a live image display region 610, and an image parameter input region 620. The live image display region 610 is a display region for the live image. If the image mode is the C mode, the live image display region 610 includes a B mode live image display region 611 for displaying the B mode live image, and a C mode live image display region 612 for displaying the C mode live image in the ROI. It is assumed that the C mode live image display region 612 can be freely moved in response to an input by the operator through the operation unit 2.

The image parameter input region 620 is an input region of the setting values of image parameters corresponding to the image mode input in step S12. For the image parameters, for example, the image parameter input region 620 includes an image parameter value display region 621 for displaying the setting values, a value input button 622 for accepting input of setting values, a page switching button 623 of the image parameter input region 620, and a close button 624.

In step S14, the operator brings the ultrasonic probe 101 into contact with a desired position of the subject. At this time, the controller 9 causes the transmitter 3 to emit a drive signal for displaying the live image in the image mode input in step S12 to the ultrasonic probe 101 in contact with the subject, causes this probe to emit the transmission ultrasonic waves, causes the receiver 4 to receive a reception signal corresponding to reflected ultrasonic waves (echo) from the ultrasonic probe 101, causes the B mode image generator 5, or the B mode image generator 5 and the C mode image generator 7 or the other mode image generator 12 to generate ultrasonic image data, causes the display processor 8 to combine the data appropriately, and causes the display 11 to display the ultrasonic image of the live image of the subject.

On the image parameter set creation screen 600, the value of the dynamically associated depth is changed by a dial input or a trackball operation, the controller 9 appropriately controls the transmitter 3, the receiver 4, the B mode image generator 5, the ROI setter 6, the C mode image generator 7, the other mode image generator 12, the display processor 8 and the like, in association with the input value of the dynamically associated depth and the setting values of the image parameters, every time a key input or a touch input is made through the value input button 622, and causes the live image display region 610 to display the live image according to the input value of the dynamically associated depth and the setting values of the image parameters.

The value of the dynamically associated depth is input through a dial input or a trackball operation through the operation unit 2 by the operator in step S14, and every time the setting values of image parameters are input through a key input or a touch input on the value input button 622, the controller 9 holds the input value of the dynamically associated depth and the setting values of image parameters (step S15). It is then determined whether another value of the dynamically associated depth or the setting value of an image parameter is input from the operator through the operation unit 2 or not (step S16). If the other value of the dynamically associated depth or the setting value of the image parameter is input (step S16; YES), the processing transitions to step S14.

If the set is changed (step S13; NO), the controller 9 reads, from the storage 10, the image mode input in step S12 and the image parameter set corresponding to the dynamically associated depth, displays list information about the read image parameter set on the display 11, and accepts a selection of the image parameter set from the list information by the operator through the operation unit 2 (step S17).

The controller 9 generates the image parameter set creation screen information corresponding to the image parameter set selected in step S17 and displays the information on the display 11, accepts, through the operation unit 2, an input of the value of the dynamically associated depth to be changed, and the setting values of the image parameters in the image mode input in step S12 in association with the dynamically associated depth, and reflects the input information (the value of the dynamically associated depth, and the setting value of the image parameter in the image mode), into the live image displayed in the image parameter set creation screen information (step S18). In step S18, for example, on the displayed image parameter set creation screen 600, the setting value of each image parameter in the image parameter set selected in step S17 is displayed in the image parameter value display region 621, the depth is changed by a dial input through the operation unit 2, and every time a key input or a touch input is performed on the value input button 622, the controller 9 displays the live image according to the input dynamically associated depth and the setting values of the image parameters, in the live image display region 610, in a manner analogous to that in step S14.

The value of the dynamically associated depth is input through a dial input or a trackball operation through the operation unit 2 by the operator in step S18, and every time the setting values of image parameters are input through a key input or a touch input on the value input button 622, the controller 9 holds the input value of the dynamically associated depth and the setting values of image parameters (step S19). The controller 9 then determines whether another value of the dynamically associated depth or the setting value of an image parameter is input from the operator through the operation unit 2 or not (step S20). If the other value of the dynamically associated depth or the setting value of the image parameter is input (step S20; YES), the processing transitions to step S18.

Steps S14 to S16 and S18 to S20 are for input of setting values of image parameters with respect to each value of the dynamically associated depth. The same setting value may be set for image parameters with different values of the dynamically associated depths. Accordingly, a configuration may be adopted where in steps S14 and S18, the image parameter copy window 700 shown in FIG. 10 is displayed on the display 11, and the same setting value of image parameters may be copied to different depths.

The image parameter copy window 700 includes a total shallow copy button 710, a total deep copy button 720, a one-step shallower copy button 730, a one-step deeper copy button 740, a save button 750, an end button 760, a setting confirmation button 770, a selected image parameter total shallow copy button 780, and a selected image parameter total deep copy button 790.

The total shallow copy button 710 is a button for accepting an input for execution of copying the setting values of image parameters at the selected dynamically associated depth to the setting values of the image parameters at all the dynamically associated depth values smaller than the value at the dynamically associated depth. For example, in the image parameter set 210 in FIG. 5, if the total shallow copy button 710 is key-input or touch-input during the ROI depth of 2 to 3 [cm] as the dynamically associated depth being selected, setting values of the image parameters at the ROI depth of 2 to 3 [cm] are held so as to be copied to the setting values of the image parameters at smaller ROI depths of 0 to 2 [cm] as shown in FIG. 11A.

The total deep copy button 720 is a button for accepting an input for execution of copying the setting values of image parameters at the selected dynamically associated depth to the setting values of the image parameters at all the dynamically associated depth values larger than the value at the dynamically associated depth. For example, in the image parameter set 210 in FIG. 5, if the total deep copy button 720 is key-input or touch-input during the ROI depth of 3 to 4 [cm] as the dynamically associated depth being selected, setting values of the image parameters at the ROI depth of 3 to 4 [cm] are held so as to be copied to the setting values of the image parameters at larger ROI depths of 4 to 8 [cm] or more as shown in FIG. 11A.

The one-step shallower copy button 730 is a button for accepting an input for execution of copying the setting values of image parameters at a dynamically associated depth value one-step shallower than the selected dynamically associated depth value to the setting values of the image parameters at the selected dynamically associated depth value. For example, in the image parameter set 210 in FIG. 5, if the one-step shallower copy button 730 is key-input or touch-input during the ROI depth of 2 to 3 [cm] as the dynamically associated depth being selected, setting values of the image parameters at the ROI depths of 1 to 2 [cm], which are shallower by one step than the ROI depths of 2 to 3 [cm], are held so as to be copied to the setting values of the image parameters at the ROI depths of 2 to 3 [cm].

The one-step deeper copy button 740 is a button for accepting an input for execution of copying the setting values of image parameters at a dynamically associated depth value one-step deeper than the selected dynamically associated depth value to the setting values of the image parameters at the selected dynamically associated depth. For example, in the image parameter set 210 in FIG. 5, if the one-step deeper copy button 740 is key-input or touch-input during the ROI depth of 3 to 4 [cm] as the dynamically associated depth being selected, setting values of the image parameters at the ROI depths of 4 to 6 [cm], which are deeper by one step than the ROI depths of 3 to 4 [cm], are held so as to be copied to the setting values of the image parameters at the ROI depths of 3 to 4 [cm].

The selected image parameter total shallow copy button 780 is a button for accepting an input for execution of copying the setting value of the selected image parameter at the selected dynamically associated depth to the setting value of the selected image parameter at every dynamically associated depth value smaller than the value at the dynamically associated depth. For example, in the image parameter set 210 in FIG. 5, if the selected image parameter total shallow copy button 780 is key-input or touch-input during the number of color Doppler repetitive transmissions being selected as the image parameter and the ROI depths of 2 to 3 [cm] as the dynamically associated depths being selected, setting values of the number of color Doppler repetitive transmissions at the ROI depths of 2 to 3 [cm] are held so as to be copied to the setting values of the number of color Doppler repetitive transmissions at smaller ROI depths of 0 to 2 [cm] as shown in FIG. 12.

The selected image parameter total deep copy button 790 is a button for accepting an input for execution of copying the setting value of the selected image parameter at the selected dynamically associated depth to the setting value of the selected image parameter at every dynamically associated depth value larger than the value at the dynamically associated depth. For example, in the image parameter set 210 in FIG. 5, if the total shallow copy button 710 is key-input or touch-input during the number of color Doppler repetitive transmissions being selected as the image parameter and the ROI depths of 3 to 4 [cm] as the dynamically associated depths being selected, setting values of the number of color Doppler repetitive transmissions at the ROI depths of 3 to 4 [cm] are held so as to be copied to the setting values of the number of color Doppler repetitive transmissions at larger ROI depths of 4 to 8 or larger [cm] as shown in FIG. 12.

The save button 750 is a button for accepting an input for execution of saving the input image parameter set. The end button 760 is a button for accepting an input for execution of finishing the image parameter set creation process. The setting confirmation button 770 is a button for accepting an input for execution of displaying the image parameter saved in step S15 or S19 on the display 11 for confirmation.

A configuration may be adopted where in steps S14 and S18, the controller 9 displays the table of the selected image parameter set on the display 11, and accepts an input of changing the value of the dynamically associated depth and the setting values of the image parameters in the displayed table of the image parameter set.

Returning to FIG. 8, if another dynamically associated depth value or the setting values of the image parameters are not input (step S16; NO) or (step S20; NO), the controller 9 determines whether save execution has been input from the operator through the operation unit 2 or not (step S21). In step S21 after step S20, for example, determination is performed according to whether or not the save button 750 has been subjected to key input or touch input. If the save execution has not been input (step S21; NO), the image parameter set creation process is finished.

If the save execution has been input (step S21; YES), the controller 9 accepts an input of the name of the image parameter set and the tag to be newly created or changed, from the operator through the operation unit 2 (step S22).

The controller 9 then saves, in the storage 10, the image parameter set that is multiple image parameters of the dynamically associated depth values held in step S15 or S19, in association with the image mode input in step S12, and the name and the tag input in step S22 (step S23), and finishes the image parameter set creation process. A configuration may be adopted where in step S23, the image parameter set to be saved is displayed on the display 11.

A configuration may be adopted where in steps S18 to S20 for the image parameter set creation process, the operator can identify (designate and input, through the operation unit 2) the image parameter to be changed and the image parameter to be maintained (not changed) in the selected image parameter set, and input only the setting value of the image parameter to be changed and actually change it.

Next, referring to FIGS. 13 to 14B, the default image parameter set setting process is described. The default image parameter set setting process is a process of selecting and setting the default image parameter set in each image mode to be associated with a desired preset (for example, the setting group corresponding to a test site (diagnostic site) of the subject, and including the setting values of image parameters that are not dynamically associated with the dynamically associated depth, and the depth value of the default dynamically associated depth).

It is assumed that in the ultrasonic diagnostic apparatus 1, through preliminary execution of the image parameter set creation process, the image parameter set (the image parameter set group) in each image mode is created and stored in the storage 10.

In the ultrasonic diagnostic apparatus 1, for example, an input of an execution instruction for the default image parameter set setting process from the operator through the operation unit 2 serves as a trigger, and the controller 9 executes the default image parameter set setting process according to the default image parameter set setting program stored in the ROM.

As shown in FIG. 13, first, the controller 9 accepts a selection of a predetermined preset from the operator through the operation unit 2 (step S31). The controller 9 then creates the image parameter set setting screen information corresponding to the preset selected in step S31, and displays the information on the display 11 (step S32). In step S32, for example, the image parameter set setting screen 800 shown in FIG. 14A is displayed. The image parameter set setting screen 800 includes an image mode display region 810, a set image parameter set display region 820, a list button 830, and an end setting button 840.

The image mode display region 810 is a region for displaying multiple image modes. The set image parameter set display region 820 is a region for displaying the name of the default image parameter set that has already been set and correspond to each image mode of the image mode display region 810. Without setting, “No” is displayed. The list button 830 is a button that is provided for each image mode of the image mode display region 810, and accepts an input of an instruction of displaying the image parameter set list screen. The end setting button 840 is a button for accepting an input for finishing setting of the default image parameter set.

In response to the image parameter set setting screen information displayed in step S32, the controller 9 accepts, through the operation unit 2 from the operator, a selection of the image parameter set in each image mode corresponding to the preset selected in step S11 through the operation unit 2 (step S33). In step S33, through a key input or a touch input on the list button 830 in the image parameter set setting screen 800, the image parameter set selection window 900 shown in FIG. 14B is displayed, for example. The image parameter set selection window 900 includes a name display region 911, a tag 1 display region 912, a tag 2 display region 913, a selection button 920, an edit button 930, a delete button 940, a non-select button 950, and a close button 960.

The name display region 911 is a display region for a list of names of image parameter sets corresponding to the image mode of the key-input or touch-input list button 830. The tag 1 display region 912 is a display region for the list of tags 1 corresponding to the image parameter sets displayed in the name display region 911. The tag 2 display region 913 is a display region for the list of tags 2 corresponding to the image parameter sets displayed in the name display region 911. The selection button 920 is a button for accepting an input of execution of selecting (setting) the image parameter set selected from the list of image parameter sets displayed in the name display region 911.

The edit button 930 is a button for accepting an input of editing the image parameter set selected from the list of the image parameter sets displayed in the name display region 911. For example, the image parameter set is edited according to processes analogous to those in steps S18 to S23 in the image parameter set creation process in FIG. 8. The delete button 940 is a button for accepting an input for deleting, from the storage 10, the image parameter set selected from the list of image parameter sets displayed in the name display region 911. The non-select button 950 is a button for accepting an input for not selecting (setting) from the list of the image parameter sets displayed in the name display region 911. The close button 960 is a button for accepting an input for execution of closing the image parameter set selection window 900. Upon input of the close button 960, the display returns to the display of the image parameter set setting screen 800.

Returning to FIG. 13, the controller 9 determines whether or not the end setting button 840 is pressed and input by the operator through the operation unit 2 (step S34). If the end setting button 840 is not pressed (step S34;

NO), the processing transitions to step S33. If the end setting button 840 is pressed (step S34; YES), the controller 9 sets the default image parameter set in each image mode selected in step S32 in association with the preset selected in step S31 (step S35), and finishes the default image parameter set setting process. In step S35, for example, the name of the default image parameter set in each image mode is stored in the storage 10 in association with the preset.

A configuration may be adopted where in step S31, the desired (type of) the ultrasonic probe, or the preset and the (type of) ultrasonic probe are input, and in step S35, the default image parameter set in each image mode selected in step S32 is set in association with the (type of) the ultrasonic probe, or the preset and the (type of) ultrasonic probe input in step S31.

Next, referring to FIGS. 15 and 16, the C mode image parameter set application process is described. The C mode image parameter set application process is a process of generating and displaying the C mode image data during the test for the subject in the C (Color Doppler) mode, and of applying the setting value of the image parameter set corresponding to the desired type of depth for C mode image data generation (the display depth, the ROI depth or the focus depth in the C mode). Here, for the sake of simplicity, a case where the dynamically associated depth (the target depth to be dynamically associated with the image parameter set) desired by the operator is the ROI depth is described. However, the case is not limited thereto. Another configuration may be adopted where the dynamically associated depth is the focus depth or the display depth, or the depth is appropriately changed such that the ROI depth ⇔ the focus depth ⇔ the display depth.

In the ultrasonic diagnostic apparatus 1, for example, an input of an execution instruction for the C mode image parameter set application process serves as a trigger, and the controller 9 executes the C mode image parameter set application process according to the C mode image parameter set application program stored in the ROM.

As shown in FIG. 15, first, the controller 9 accepts a selection of the C mode test preset (the test site or the like) from the operator through the operation unit 2 (step S41). Accordingly, the depth (here, the ROI depth) is set to the depth value set by the preset.

The controller 9 then refers to the name of the default image parameter set with respect to each preset stored in the storage 10, and enables the default image parameter set (a first image parameter set) corresponding to the preset selected in step S41 (step S42). If the image parameter set in the C mode is set in association with the (type of) ultrasonic probe, the controller 9 obtains type information (for example, stored in a memory of a connector of the cable 102) on the ultrasonic probe 101 connected to the ultrasonic diagnostic apparatus main body 100 in step S41. In step S42, the (type of) ultrasonic probe input in step S41 or the default image parameter set (assumed as the first image parameter set) corresponding to the preset or the (type of) the ultrasonic probe is enabled.

The operator starts to bring the ultrasonic probe 101 into contact with a desired position on the subject. In response to an input for turning on the C (color Doppler) mode from the operator through the operation unit 2, the controller 9 then turns on the C mode (step S43). The controller 9 sets or changes the setting value of the image parameter that is not dynamically associated with the ROI depth of the first image parameter enabled in step S42, to the setting value of the image parameter that is not dynamically associated with the ROI depth to be applied to C mode image data to be generated (step S44).

The controller 9 sets or changes the setting value of the image parameter dynamically associated with the ROI depth of the first image parameter set enabled in step S42, to the setting value of the image parameter dynamically associated with the ROI depth to be applied to C mode image data to be generated (step S45).

The controller 9 then controls the transmitter 3, the receiver 4, the B mode image generator 5, the ROI setter 6, the C mode image generator 7 and the display processor 8 according to the preset ROI depth or the input ROI position (ROI depth), the setting value of the image parameter that is in the first image parameter set set in step S42 and is not dynamically associated with the ROI depth, and the setting value of the image parameter that is in the first image parameter set set in step S43 and is dynamically associated with the ROI depth, generates and combines the B mode image data and the C mode image data, and starts to display the combined image data as the live image on the display 11 (step S46).

The displayed C mode image based on the C mode image data can freely change the ROI position according to an input by the operator through the operation unit 2.

The controller 9 then determines whether an input for ROI movement from the operator through the operation unit 2 is present or not (step S47). If the input for ROI movement is present (step S47; YES), the processing transitions to step S45.

If the input for ROI movement is not present (step S47; NO), the controller 9 determines whether an input for application of a second image parameter set from the operator through the operation unit 2 is present or not (step S48). In step S48, for example, if a request for selecting the second image parameter set is made from the operator through the operation unit 2, the controller 9 reads, from the storage 10, the image parameter set that corresponds to the C mode and is different from the first image parameter set, displays a button for accepting a selection of the read image parameter set on the display 11, accepts a click input or a touch input on the button for the displayed image parameter set from the operator through the operation unit 2, and adopts the input as an input for application as the second image parameter set.

If the input for application of the second image parameter set is not present (step S48; NO), the controller 9 determines whether or not to finish the test of the subject according to presence or absent of an input for finishing the test from the operator through the operation unit 2 (step S49). If the test is not to be finished (step S49; NO), the processing transitions to step S46. If the test is to be finished (step S49; YES), the C mode image parameter application process is finished.

If an input for application of the second image parameter set is present (step S48; YES), the controller 9 enables the second image parameter set input in step S48 (step S50). At this time, the image mode is not changed and remains to be C mode.

The controller 9 sets or changes the setting value of the image parameter that is not dynamically associated with the ROI depth of the second image parameter enabled in step S50, to the setting value of the image parameter that is not dynamically associated with the ROI depth to be applied to C mode image data to be generated (step S51).

The controller 9 sets or changes the setting value of the image parameter dynamically associated with the ROI depth of the second image parameter set enabled in step S50, to the setting value of the image parameter dynamically associated with the ROI depth to be applied to C mode image data to be generated (step S52).

The controller 9 then controls the transmitter 3, the receiver 4, the B mode image generator 5, the ROI setter 6, the C mode image generator 7 and the display processor 8 according to the input ROI position (ROI depth), the setting value of the image parameter that is in the second image parameter set set in step S51 and is not dynamically associated with the ROI depth, and the setting value of the image parameter that is in the second image parameter set set in step S52 and is dynamically associated with the ROI depth, generates and combines the B mode image data and the C mode image data, and starts to display the combined image data as the live image on the display 11 (step S53).

Step S54 is analogous to step S47. If the input for ROI movement is not present (step S54; NO), the controller 9 determines whether an input for turning off application of the second image parameter set from the operator through the operation unit 2 is present or not (step S55). An input for turning off application of the second image parameter set is performed by, for example, clicking or touching again the button for accepting an input of application of the second image parameter set having already been applied. If the input for turning off application of the second image parameter set is present (step S55; YES), the processing transitions to step S42. Step S56 is analogous to step S49.

As described above, when the second image parameter set is turned off (after the selection of the second image parameter set, the same selection is made again), the first image parameter set is enabled if the default first image parameter set has been registered.

For example, the following case is assumed. First, according to the default image parameter set setting process, for the preset for abdomen testing, an image parameter set generally usable for an abdomen is preliminarily registered. In the C mode image parameter set application process, a button for accepting a selection of the second image parameter set dedicated for an aorta is displayed in the display screen on the display 11. If a test is intended for the aorta, the operator performs a click input or a touch input on the button for selecting the second image parameter set for the aorta through the operation unit 2. After the test for the aorta is finished, the operator performs a click input or a touch input again the button for selecting the second image parameter set for an aorta, thus turning off the selection of the second image parameter set. At this time, in the case where the default image parameter has been registered, an operation of returning to the first image parameter set, which is the default image parameter set generally applicable to an abdomen, is desirable for the operator's usability, rather than bringing the state to a state completely without application of the image parameter set.

The test sites of the first image parameter set and the second image parameter set may be the same as each other. For example, the first image parameter set may be an image parameter set generally usable for an abdomen, and the second image parameter set may be an image parameter set that is for a fat subject, has high penetration and is usable for an abdomen.

The configuration is thus adopted where in the C mode, the C mode image parameter set application process applies the image parameter set to the C mode image data to be generated. However, the configuration is not limited thereto. A process may be adopted where in the C mode, the image parameter set is applied to the C mode image data to be generated, and the image parameter set is applied to the B mode image data to be generated at the same time.

Also in other modes (the elastography mode, the B mode, the M mode, the pulse wave Doppler mode, etc.), as with the C mode image parameter set application process, the default first image parameter set, and the desired second image parameter set are applied to generation of ultrasonic image data.

As described above, according to this embodiment, the ultrasonic diagnostic apparatus 1 includes: the image generator (the transmitter 3, the receiver 4, the B mode image generator 5, the ROI setter 6, the C mode image generator 7, the display processor 8, and the other mode image generator 12) that generates ultrasonic image data on the basis of a reception signal obtained by an ultrasonic probe transmitting and receiving ultrasonic waves to and from a subject; the storage 10 that stores first image parameters (image parameter set) associated with the depth corresponding to the ROI; and the controller 9 that causes the image generator to generate the ultrasonic image data on the basis of the first image parameters.

Accordingly, in the image mode for displaying the ultrasonic image in the ROI, appropriate image parameters can be applied in conformity with the ROI depth of the ultrasonic image, and appropriate ultrasonic image data can be generated.

The ultrasonic diagnostic apparatus 1 includes the color Doppler mode and the elastography mode, as the image modes. The controller 9 includes the color Doppler mode and the elastography mode as the image modes. The controller 9 causes the image generator to generate ultrasonic image data in the color Doppler mode or the elastography mode, on the basis of the first image parameters. Accordingly, in the color Doppler mode or the elastography mode as the image mode for displaying the ultrasonic image in the ROI, an appropriate image parameter can be applied in conformity with the ROI depth of the ultrasonic image, and appropriate ultrasonic image data can be generated.

The depth corresponding to the ROI is an ROI depth corresponding to the position at a predetermined ratio between the upper end and the lower end in the depth direction of the ROI. Accordingly, an appropriate image parameter corresponding to an appropriate ROI depth can be applied. For example, the ROI depth may be a depth positioned at an exactly intermediate distance between the upper end and the lower end in the ROI depth direction, or a depth at another position between the upper end and the lower end according to setting by the operator.

The depth corresponding to the ROI is the focus depth. The focus depth is a focus depth dynamically associated with the ROI depth. Accordingly, an appropriate image parameter corresponding to an appropriate focus depth can be applied.

The first image parameters include the transmission frequency, the number of repetitive transmissions, the sound ray density, the time average, and the frame rate priority setting. Accordingly, an appropriate image parameter corresponding to the ROI depth or the focus depth can be applied.

The controller 9 causes the image generator to generate ultrasonic image data in the color Doppler mode or the elastography mode, on the basis of the first image parameters dynamically associated with the ROI depth or the focus depth, and the second image parameter that is not dynamically associated with the ROI depth or the focus depth. Accordingly, besides the first image parameters associated with the ROI depth or the focus depth, the second image parameter that is preferably not dynamically associated with the ROI depth or the focus depth can also be applied, and more appropriate ultrasonic image data can be generated.

The storage 10 stores a plurality of image parameter sets including the first image parameters. The ultrasonic diagnostic apparatus 1 further includes the operation unit 2 that accepts a selection of an image parameter set from among the image parameter sets. The controller 9 causes the image generator to generate ultrasonic image data in the color Doppler mode or the elastography mode, on the basis of the first image parameters in the selected image parameter set. Accordingly, the desired image parameter set can be selected. The image parameters in the selected image parameter set can be applied. More appropriate ultrasonic image data can be generated.

The storage 10 stores a plurality of image parameter sets including the first image parameters associated with each ROI depth or each focus depth. The controller 9 causes the image generator to generate ultrasonic image data on the basis of the first image parameters associated with the depth corresponding to the ROI. Accordingly, over ROI depths or focus depths, an appropriate image parameter can be applied, and more appropriate ultrasonic image data can be generated.

The image parameter set may include a second image parameter different from the first image parameters.

The second image parameter may be an image parameter that is not dynamically associated with the ROI depth or the focus depth. Accordingly, the first image parameters associated with the ROI depth or the focus depth, and the image parameter that is preferably not dynamically associated with the ROI depth or the focus depth are associated with each other, and can be easily read and applied, thereby allowing more appropriate ultrasonic image data to be generated.

The image parameters that are not dynamically associated with the ROI depth or the focus depth include the flow velocity scale, the steering angle, and the ROI size. Accordingly, the flow velocity scale, the steering angle, and the ROI size that are appropriate as the image parameters dynamically associated with the ROI depth or the focus depth can be applied.

The ultrasonic diagnostic apparatus 1 includes the pulse wave Doppler mode and the continuous wave Doppler mode, as the image modes. The storage 10 stores third image parameters associated with the gate depth or the focus depth. The controller 9 causes the image generator (the transmitter 3, the receiver 4, the B mode image generator 5, the other mode image generator 12, and the display processor 8) to generate ultrasonic image data in the pulse wave Doppler mode or the continuous wave Doppler mode on the basis of the third image parameters. Accordingly, in the pulse wave Doppler mode or the continuous wave Doppler mode, an appropriate image parameter in conformity with the gate depth or the focus depth can be applied, and appropriate ultrasonic image data can be generated.

The third image parameters include the transmission frequency. Accordingly, a transmission frequency appropriate as an image parameter dynamically associated with the gate depth or the focus depth can be applied.

The ultrasonic diagnostic apparatus 1 includes the B mode and the M mode, as the image modes. The storage 10 stores fourth image parameters associated with the display depth. The controller 9 causes the image generator (the transmitter 3, the receiver 4, the B mode image generator 5, and the display processor 8) to generate ultrasonic image data in the B mode or the M mode on the basis of the fourth image parameters. Accordingly, in the B mode or the M mode, an appropriate image parameter in conformity with the display depth can be applied, and appropriate ultrasonic image data can be generated.

The operation unit 2 accepts an input of an image parameter dynamically associated with the depth with respect to each of the image modes, and the controller 9 creates an image parameter dynamically associated with each input image mode input. Accordingly, the image parameter dynamically associated with the depth in each image mode can be easily created.

The operation unit 2 accepts an input of a display depth, an ROI depth or a focus depth, as a depth dynamically associated with the image parameters in the image modes. The controller 9 creates the image parameter dynamically associated with the display depth, the ROI depth or the focus depth in each image mode input. Accordingly, the image parameter dynamically associated with the display depth, the ROI depth or the focus depth in each image mode can be easily created.

The image modes include the B mode, the M mode, the color Doppler mode, the elastography mode, the pulse wave Doppler mode, and the continuous wave Doppler mode. Accordingly, the image parameter dynamically associated with the depth (the display depth, the ROI depth or the focus depth) in the B mode, the M mode, the color Doppler mode, the elastography mode, the pulse wave Doppler mode, and the continuous wave Doppler mode can be easily created.

The operation unit 2 accepts an input of presence or absence of application of the image parameter to each of the image modes. The controller 9 sets presence or absence of application of the image parameter in each image mode input. Accordingly, presence or absence of application of the image parameter in each image mode can be easily set.

The controller 9 registers the first image parameter set (including the first image parameters and the second image parameter) as the default image parameter. In a case where application of the second image parameter set (including the first image parameters and the second image parameter) is turned off and the default image parameter is registered, the controller 9 causes the image generator to generate ultrasonic image data on the basis of the default image parameters. Accordingly, for example, when application of the dedicated second image parameter set is turned off, multiple image parameters in the general default image parameter set can be easily applied, appropriate ultrasonic image data can be generated, and the operability can be improved.

The operation unit 2 accepts an input for turning on application of the second image parameter set. If, in a state where an input for turning on application of the second image parameter set is performed and the application of the second image parameter set is turned on, the same input is performed again, the controller 9 turns off the application of the second image parameter set and turns on application of the default image parameter set. Accordingly, the input for turning on the application of the second image parameter set is performed twice, which can easily turn on application of multiple default image parameters, thereby allowing the operability to be improved.

The controller 9 registers the default image parameters in association with at least one of the preset and the ultrasonic probe. The controller 9 turns on application of the default image parameters corresponding to at least one of the designated preset and ultrasonic probe. Accordingly, application of appropriate image parameters associated with at least one of the preset and the ultrasonic probe can be turned on.

The description of the embodiments and modification examples is only an example of an appropriate ultrasonic diagnostic apparatus according to the present invention, and is not limited thereto.

For example, according to the embodiments described above, the image parameter set has the configuration where the setting value of the image parameter dynamically associated with (corresponding to) one type of depth (the ROI depth, the focus depth or the display depth) is stored. However, the configuration is not limited thereto. The image parameter set may be configured such that setting values of image parameters corresponding to multiple types of depths are stored. For example, a configuration may be adopted where in the image parameter set corresponding to the C mode, the setting value of each image parameter dynamically associated with (corresponding to) the ROI depth and the display depth is stored. In this configuration, for example, according to the C mode image parameter set application process in FIG. 15, in step S45 or S52, setting value of the image parameter dynamically associated with the ROI depth and the display depth in the first or the second image parameter set is set to the image parameter dynamically associated with the ROI depth and the display depth for generating ultrasonic image data. Accordingly, even in a case where the ROI is positioned at a shallow depth during observation at a shallow site, the display size (scale) of the image is different depending on the display depth. Consequently, even in a case where the optimal image parameter is changed, an appropriate image parameter can be applied, and appropriate ultrasonic image data can be generated. For example, the data density in the depth direction is different. Accordingly, it can be considered that the filter coefficients in the depth direction as the image parameters are changed in conformity with the ROI depth and the display depth.

Detailed configurations and detailed operations of the components constituting the ultrasonic diagnostic apparatus 1 in the above embodiment can be appropriately changed without departing from the spirit of the present invention.

Although embodiments of the present invention have been described and illustrated in detail, the disclosed embodiments are made for purposes of illustration and example only and not limitation. The scope of the present invention should be interpreted by terms of the appended claims

The entire disclosure of Japanese patent Application No.2018-207150, filed on 2nd of Nov. 2018, is incorporated herein by reference in its entirety.

What is claimed is:

1. An ultrasonic diagnostic apparatus, comprising:
a first hardware processor that generates ultrasonic image data, based on a reception signal obtained by an ultrasonic probe that transmits and receives an ultrasonic wave;
a storage that stores first image parameters associated with a depth corresponding to an ROI; and
a second hardware processor that causes the first hardware processor to generate the ultrasonic image data, based on the first image parameters.

2. The ultrasonic diagnostic apparatus according to claim 1, including
at least one of a color Doppler mode and an elastography mode as an image mode,
wherein the second hardware processor causes the first hardware processor to generate the ultrasonic image data in the color Doppler mode or the elastography mode, based on the first image parameters.

3. The ultrasonic diagnostic apparatus according to claim 1, wherein the depth corresponding to the ROI is an ROI depth corresponding to a position at a predetermined ratio between an upper end and a lower end in a depth direction of the ROI.

4. The ultrasonic diagnostic apparatus according to claim 1,
wherein the depth corresponding to the ROI is a focus depth, and
the focus depth is dynamically associated with an ROI depth corresponding to a position at a predetermined ratio between an upper end and a lower end in a depth direction of the ROI.

5. The ultrasonic diagnostic apparatus according to claim 1, wherein the first image parameters are associated with a combination of a depth corresponding to the ROI and a display depth.

6. The ultrasonic diagnostic apparatus according to claim 1, wherein the first image parameters include at least one of a transmission frequency, the number of repetitive transmissions, a sound ray density, a time average, and frame rate priority setting.

7. The ultrasonic diagnostic apparatus according to claim 1, wherein the second hardware processor causes the first hardware processor to generate the ultrasonic image data, based on the first image parameters and a second image parameter that is not dynamically associated with the depth corresponding to the ROI.

8. The ultrasonic diagnostic apparatus according to claim 1,
wherein the storage stores a plurality of image parameter sets including the first image parameters,
the apparatus further comprises a first operation unit that accepts a selection of an image parameter set from among the image parameter sets, and
the second hardware processor causes the first hardware processor to generate the ultrasonic image data, based on the image parameters in the selected image parameter set.

9. The ultrasonic diagnostic apparatus according to claim 1,
wherein the storage stores a plurality of image parameter sets including the first image parameters associated with the depth corresponding to the ROI, and
the second hardware processor causes the first hardware processor to generate the ultrasonic image data, based on the first image parameters associated with the depth corresponding to the ROI.

10. The ultrasonic diagnostic apparatus according to claim 8, wherein the image parameter set includes a second image parameter different from the first image parameters.

11. The ultrasonic diagnostic apparatus according to claim 10, wherein the second image parameter is an image parameter that is not dynamically associated with the depth corresponding to the ROI.

12. The ultrasonic diagnostic apparatus according to claim 11, wherein the image parameter that is not dynamically associated with the depth corresponding to the ROI is at least one of a flow velocity scale, a steering angle, and an ROI size.

13. The ultrasonic diagnostic apparatus according to claim 1, including
at least one of a pulse wave Doppler mode and a continuous wave Doppler mode,
wherein the storage stores a third image parameters associated with a gate depth or a focus depth, and
the second hardware processor causes the first hardware processor to generate the ultrasonic image data in the pulse wave Doppler mode or the continuous wave Doppler mode, based on the third image parameters.

14. The ultrasonic diagnostic apparatus according to claim 13, wherein the third image parameters include a transmission frequency.

15. The ultrasonic diagnostic apparatus according to claim 1, including
at least one of a B mode and an M mode,
wherein the storage stores a fourth image parameters associated with a display depth, and
the second hardware processor causes the first hardware processor to generate the ultrasonic image data in the B mode or the M mode, based on the fourth image parameters.

16. The ultrasonic diagnostic apparatus according to claim 1, further comprising a second operation unit that accepts input of an image parameter dynamically associated with the depth for each of the image modes, and
wherein the second hardware processor creates the input image parameter dynamically associated with the depth in each image mode.

17. The ultrasonic diagnostic apparatus according to claim 16,
wherein the second operation unit accepts an input of a display depth, an ROI depth or a focus depth, as a depth dynamically associated with the image parameters in the image modes, and
wherein the second hardware processor creates an image parameter dynamically associated with the input display depth, ROI depth or focus depth in each image mode.

18. The ultrasonic diagnostic apparatus according to claim 16, wherein the image modes include at least one of a B mode, an M mode, a color Doppler mode, an elastography mode, a pulse wave Doppler mode, and a continuous wave Doppler mode.

19. The ultrasonic diagnostic apparatus according to claim 16, further comprising:
a third operation unit that accepts input of presence or absence of application of the image parameter to each of the image modes, and
a setter that is provided for the second hardware processor and sets the input presence or absence of application of the image parameter in each image mode.

20. An ultrasonic diagnostic apparatus, comprising:
a first hardware processor that generates ultrasonic image data, based on a reception signal obtained by an ultrasonic probe that transmits and receives an ultrasonic wave to and from a subject;
a storage that stores first image parameters associated with a depth; and
a second hardware processor that causes the first hardware processor to generate the ultrasonic image data, based on the first image parameters, and registers default image parameters,
wherein when application of the first image parameters is turned off and the default image parameters are registered, the second hardware processor causes the first hardware processor to generate the ultrasonic image data, based on the default image parameters.

21. The ultrasonic diagnostic apparatus according to claim 20, further comprising a fourth operation unit that accepts an input for turning on application of the first image parameters,
wherein in a state where the input for turning on application of the first image parameters is performed and the application of the first image parameters is turned on, when an identical input is performed again, the second hardware processor turns off application of the first image parameters, and turns on application of the default image parameters.

22. The ultrasonic diagnostic apparatus according to claim 20, wherein the second hardware processor registers the default image parameters in association with at least one of a preset and an ultrasonic probe, and turns on or off application of the default image parameters associated with at least designated one of the preset and the ultrasonic probe.

23. The ultrasonic diagnostic apparatus according to claim 20, wherein the depth is at least one of a display depth, an ROI depth and a focus depth.
